(19) United States
(12) Patent Application Publication
(10) Pub. No.: US 2023/0398338 A1
(43) Pub. Date: Dec. 14, 2023

(54) Fluid drain control apparatus, systems, and methods

(71) Applicant: Marshall E. Fryman, Libertyville, IL (US)

(72) Inventor: Marshall E. Fryman, Libertyville, IL (US)

(21) Appl. No.: 18/053,097

(22) Filed: Nov. 7, 2022

Related U.S. Application Data
(63) Continuation-in-part of application No. 17/840,572, filed on Jun. 14, 2022.

(51) Int. Cl. A61M 27/00; A61M 1/00

(52) U.S. Cl. CPC A61M 27/006; A61M 1/70

(57) ABSTRACT

Described herein is a safety system that works collectively with an automated fluid drain control apparatus and systems and clinical experts to establish protocols and methods for given patient populations to ensure that the drainage of fluid from patients is both safe and effective. It further enables the transportation of drain orders from systems external to the drain system and returns to them the drainage data on a periodic basis for inclusion into the patient chart.

### FIELD OF THE INVENTION

The presently disclosed subject matter relates to providing apparatus, systems, and methods for management of cerebrospinal fluid, and more particularly, to apparatus, systems, and methods for drainage, analysis, and control of cerebrospinal fluid.

### BACKGROUND OF THE INVENTION

Cerebrospinal Spinal Fluid (CSF) management involves the application of devices such as shunts, valves and external drainage systems to optimize the volume and/or pressure in the intracranial spaces and drain excess CSF as needed. CSF management is widely used in the treatment of traumatic brain injury (including surgery), hydrocephalus and neurological disorders such as Parkinson's and Alzheimer's disease. Drainage of cerebrospinal fluid from a patient is traditionally performed using one

of two methods: lumbar drainage and ventricular drainage. The methods for accessing the CSF fluid involves the introduction of a catheter into the patient's subdural space. For lumbar drains, this is done between L5-S1 (e.g., intrathecal sack) spinal columns similar to how an epidural is inserted into the spine. For ventricular drains, the catheter is introduced surgically through the skull. In both cases, the catheter is then connected to a drainage system that allow some form of control of the amount of fluid drained from the patient. The physician then orders CSF to be drained either at a fixed rate per hour (lumbar drains) or when the intracranial pressure exceeds a given amount (traditionally represented in either mmHg or cmH2O). Relevant schematic representations of typical human anatomy and placement and use of catheters are shown in FIG. 1.

Body fluid drains and containers are well known in the art. For example, there are collection devices for urine and others that drain and collect spinal fluid. None of these devices are able to easily control the drainage rate of the fluid as a function of time until the introduction of U.S. Pat. No. 8,475,419, to Eckermann for a “Automated body fluid drain control apparatus and method”, expanded the art. In connection with the drainage of cerebrospinal fluid (“CSF”), for most people, the body produces 450 cc of CSF over a 24 hour period which fills the subarachnoid space in the body. There are many instances where it may be advisable and/or necessary for some of the CSF to be drained. For example, during certain medical procedures such as brain surgery, the surgeon may wish to drain some of the CSF in order to relieve pressure in the intercranial compartment. In addition, in some brain and spinal surgeries where the dura mater is penetrated, the CSF would need to be partially drained to keep pressure off the wound site in order to allow it to heal. Also, in certain head trauma cases where CSF is collecting in the cranial cavity, it may be preferable to drain some of the CSF from the subarachnoid space in the lumbar spinal region to relieve the pressure on the brain. Other patents exist in the field, including U.S. Pat. No. 9,717,890 to Holper, Traxler, Schroter, Martens, and Holper for a “Drainage system for cerebrospinal fluid”.

Conventional methods of draining CSF involve tapping into the cranial or subarachnoid space in the spinal column and draining the excess CSF through a catheter tube into a collection bag. The amount of drainage must be regulated, as if there is too much drainage, a patient can be irreversibly injured or can be fatally injured.

Unfortunately, the rate at which the CSF drains is not in a linear fashion despite production of CSF occurring at a constant rate. For example, the CSF can drain at 1 cc per hour and then suddenly drain 5 ccs in 10 minutes. Since there are irreversible and potentially fatal consequences if too much CSF is drained, the volume of the drainage has to be constantly monitored by a nurse. Due to the demand on a nurse's time and the non-linearity of the drainage, there is a potentially fatal margin of error. Thus, an apparatus that continuously monitors and controls the drainage of the CSF, as described in U.S. Pat. No. 8,475,419 provided a great benefit to the art.

### Volumetric Drainage.

As taught in U.S. Pat. No. 8,475,471, there can be a significant improvement in clinical outcome for patients when a computer-controlled drain system is utilized to automate CSF fluid drainage. Passive drainage systems rely upon the relative position of the drain system to the patient (gravity driven) and the innate pressure that the ventricular and subarachnoid space generates (including the central canal of the spinal cord) through physiological processes. This process permits overdrainage of the CSF system that is within a sub-period of the desired drainage time. For instance, a drainage at 20 cc per hour could see all 20 cc drained within seconds when the desired drainage should have occurred over one hour to align to the physiological rate of production. This results in a significant reduction of CSF pressure and volume potentially leading to hemorrhaging. Conversely, in severe trauma, a rapid reduction in pressure and volume may be desirable due to the intercranial pressure being elevated (due to overproducing CSF, edema, blood or other fluids being introduce or other physiological responses) in reaction to the trauma. Thus, a programmable bolus of volumetric drainage may be desirable to achieve an initial volumetric reduction and then return to a previously desired drainage rate. Additionally, it may be desirable to set minimum and maximum volumetric drainage limit based on patient population (e.g., pediatric vs adult patients may have different minimum and maximum volumetric drain limits), initial drainage defaults (e.g., always start at 15 mL/hr for adult patients), minimum and maximum drainage titration (change) limits (e.g., do not titrate drainage by more than 10% per instance), titration lockout periods (e.g., the user must wait at least 10 minutes between changes). Other disclosures known in the art include U.S. patent application Ser. No. 17/466,301, by Morse and Morse, for “Body Fluid Management Systems For Patient Care.”

Further, CSF fluid is routinely sampled during the procedure and requires access to the CSF fluid prior to exposure to outside contaminants such as air (oxygenation) or fluid which has been stored for some period of time (biological growth). To compound matters, the total volumetric drainage amount for a period cannot be determined without having control of the sampling means and volume sampled. It is common, in the prior art, for the CSF sample to represent over 25% of the total programmed volumetric drain amount in any one period.

### Continuous Pressure Monitoring Drainage.

In existing drainage systems, the manual system of pressure monitoring involves manually opening and closing stopcocks and utilizing a combination of fluid pressure against head height to drain the patient to maintain a given intracranial pressure (ICP). These systems have an external fluid-filled transducer that measures the ICP of the patient via the pressure at the point of transducing. Alternatively, they may measure ICP via an implantable pressure sensor in the ventricular shunt. In either case, the pressure sensor is not in communication with the manual drain and provides no feedback to the user or control of the drainage amounts. Further, because the manual drains are not in communication with the pressure sensor, the accuracy of the pressure sensor varies depending on the unknown status of the stopcock. When open, the accuracy of the pressure sensor falls off and shows a significant reduction in pressure. When closed, the accuracy returns to nominal and the pressure values being monitored suddenly return to normal. A drain that is in communication with various means of monitoring ICP can thus adjust for the open and closed state of the drain to provide normalized pressure values for standardized pressure monitoring regardless of drain state. Further, unlike a manually operated drain, an automated system can open and close the drain multiple times per minute to both achieve the targeted ICP and provide highly accurate ICP monitoring of the patient.

It is important in CSF drainage, particularly during ventricular drainage, is to ensure the device is level to or below the interventricular foramen (also known as foramen of Monro) which lies between the roof and anterior wall of the third ventricle behind the column and body of the fornix and anterior to the thalamus. This becomes the zero-reference point for “external to the ventricular system” pressure monitoring. A zero reference at external auditory meatus (EAM) or glabella is ideal at brain center (BC) when the head is strictly supine or in the lateral position. At 45° head elevation, an overestimation of the brain center-intracranial pressure (ICP) by 4.8±0.8 and in upright 5.6±0.5 mmHg was found, and 45° lateral underestimated ICP-BC by 6.3±1.0 mmHg. Monro was situated 45±5 mm rostral to the mid-orbito-meatal (OM) line and 24 (18-31) mm inferior and 13 (8-17) mm in front of BC. A zero-reference point aligned with the highest point of the head underestimates BC-ICP and Monro-ICP. If the ICP reading was added 5.9 or 6.3 mmHg, respectively, a deviation from BC-ICP was ≤1.8 mmHg and Monro-ICP was ≤0.9 mmHg in all head positions. EAM and glabella are defined anatomical structures representing BC when strictly supine or lateral but with 12 mmHg variation with different head positions used in clinical practice. The OM line follows Monro at head elevation, but not when the head is turned. When the highest external point on the head is used, ICP values at brain surface as well as Monro and BC are underestimated. This underestimation is fairly constant and, when corrected for, provides the most exact ICP reading, for example as found in “Best zero level for external ICP transducer” (Peter Reinstrup et al.; Acta Neurochir (Wien) 2019; 161(4): 635-642; accessible at https://www.ncbi.nlm.nih.govipmciarticles/PMC6431298/). It is therefore required to monitor both the base line interventricular foramen and the head position in order to ensure accuracy of the ICP pressure.

ICP waveforms can be characterized into normal and abnormal patterns. With reference to FIG. 9, a normal ICP wave form is illustrated, showing the P1 (Percussion wave), P2 (Tidal wave), and P3 (Dicrotic notch). Attempts have been made by Hammer et al to use the morphology of the ICP

pulse wave as a surrogate marker of intracranial elastance. They decided that the systolic part of the vascular ICP waveform reflects arterial activity while the caudal descending segment denotes the pressure in SVC. Hence when the ICP increases the caudal part of the ICP waveform (the P2 component) assumes the shape of an arterial pulse and when there is CVP elevation, the waveform approximates a venous pulse.

When the ICP is elevated, the vascular (cardiac) waveform amplitude increases while the respiratory waveform amplitude changes in response to the vascular increase. Other phenomena which are visible in dysfunctional intracranial compliance include occurrence of P waves as well as elevation of P2 and rounding off of the waveform. The occurrence of these phenomena are useful in clinical practice in that these alert the neurophysician to initiate ICP control measures on an urgent basis. It is pertinent to note here that increased ICP can produce characteristic waveform variously classified by Lundberg into A, B and C waves. The ICP waveform shown in FIG. 10 is indicative of intracranial hypertension.

With reference to FIG. 11, Lundberg A waves are the ones which denote highest rise in ICP (50-100 mmHg). They are generally indicative of high degree of cerebral ischemic and impending brain herniation and persist for 5 to 10 min. Lunenburg B waves occur for a lesser period of time (1 to 2 min), the ICP elevation or not as much, 20 to 30 mm Hg, and are rhythmic in nature. They indicate evolving cerebral injury causing a gradual increase in ICP. Lundberg C waves correlate with blood pressure fluctuations brought about by baroreceptors and chemoreceptor reflex mechanisms and have no clinical significance (see Nag et al., World J. Clin. Cases. Jul. 6, 2019; 7(13): 1535-1553, “Intracranial pressure monitoring: Gold standard and recent innovations”, last accessed 06/14/2021, at https://www.wjgnet.com/2307-8960/full/v7/i13/1535.htm).

An additional use case for CSF drainage is the treatment of normal pressure hydrocephalus (NPH). NPH is a clinical condition with enlarged intra-cerebral ventricles (Hakim & Adams, 1965), and symptoms of gait disturbance, enuresis and cognitive reduction (Fisher, 1982; Williams & Malm, 2016). The small step, shuffling gait is an early dominant symptom, which may be due to direct pressure on the midbrain gait center from an enlarged third ventricle (Lee, Yong, Ahn, & Huh, 2005). The gait disturbance offers an opportunity to evaluate the degree of disease progression (Chivukula et al., 2015; Williams et al., 2008), and may even help in prediction of good post-operative outcome (Gaff-Radford & Godersky, 1986).

When the underlying pathology is insufficient re-absorbtion of CSF surgical shunting of CSF to intravenous or peritoneal space may alleviate the symptoms, but the postoperative success depends on correct diagnosis. NPH coexists with, can be caused by, and may be mimicked by different forms of arteriosclerosis.

A direct diagnostic method for NPH is the constant infusion lumbar infusion test (LIT; Katzman & Hussey, 1970), where mock CSF is injected into the spinal cavity for passage through the Sylvian aqueduct intra-cranially in order to stress the CSF re-absorbtion ability (see Ryding, Kahlon, and Reinstrup, “Improved lumbar infusion test analysis for normal pressure hydrocephalus diagnosis”, Brain Behay. 2018 November; 8(11): e01125, last accessed 06/14/2021, at https://www.ncbi.nlm.nih.gov/pmc/articles/PMC6236248/).

The intent for LIT is that the lumbar infusion of mock CSF will increase the intracranial CSF volume. If the intra-cranial CSF volume increases, the only volume that can decrease in equal measure is the venous volume, since the CSF is incompressible. Likewise, the arterial blood volume delivered intra-cranially during each systole is compensated for by compression of the venous pool by the same amount. The increased venous outflow resistance due to the venous vascular compression causes an increase in intra-cranial pressure (ICP; Marmarou, Schulman, & Rosende, 1978).

The LIT test is a combination of volumetric lumbar infusion and ICP pressure monitoring. In one study, the lumbar infusion test was done using a constant infusion rate (0.80 ml/min) and regarded as positive if the steady state CSF plateau pressure reached levels of >22 mm Hg (resistance to outflow >14 mm Hg/ml/min).

Another diagnostic test for NPH is a CSF tap test (also known as: lumbar tap test, tap test, or Miller Fisher Test). This typically involves removing 30 mL of CSF through a lumbar puncture after which cognitive function is assessed.

NPH positive predictive values were 80% for lumbar infusion test and 94% for tap test. The system of the present disclosure supports both modes of drainage through its ability to interface externally with an infusion pump which is connected to a Y-site located distal to the patient but proximal to the system of the present disclosure. The infusion pump reports its infusion rates directly to the system of the present disclosure which monitors the ICP using its internal pressure transducer.

Traditionally in the prior art, cerebrospinal fluid (CSF) has been examined using a process that pulls a sample from of CSF from a patient based on an identified risk or event. The fluid is then examined in a laboratory setting to detect blood and blood products from haemorrhage. Fluid from patients with this condition will contain red blood cells unless they have been completely metabolized—an event which typically takes at least 7 days to occur. Red blood cells lyse, releasing oxyhaemoglobin which is then converted into bilirubin. After centrifugation, the CSF supernatant is visible pink or pink-orange in color from oxyhaemoglobin, yellow due to billirubin and intermediate if both are present.

With the advent of spectrophotometry, the laboratory is now able to identify data without the introduction of centrifuges and other laborious processes. It is common to identify oxyhaemoglobin (413-415 nm), oxyhaemoglobin and bilirubin (broad peak/shoulder at 450-460 nm), and bilrubin alone. Methaemlobin may also be identified (405 nm shifting to 413 nm when oxyhaemgloin is present). It is also possible to identify glucose (˜1500 nm) and insulin (˜260-350 nm) in bodily fluids and proteins at ˜1575 nm.

All of these methods rely on traditional laboratory techniques and instruments. The application occurs against the entire column of fluid and requires re-sampling each time the test needs to be run.

With the advent of electromechanical fluid drains, it now becomes possible to develop and implement clinically driven safety protocols that control and instruct the device to more safely drain body fluids from a given patient population. In today's environment, all patients are treated as identical by the device. With this enhancement to the art, the device will become aware of the unique physiological parameters of the patient and the clinical diagnosis', including comorbidities, which will instruct the device in how to properly monitor and drain the patient. Further, the same device can be customized to any given patient through the use of a set of clinical parameters that are entered by the clinician on the device to select the unique drain conditions that are applicable to this patient.

We also consider user preference and needs for novel methods of entering, selecting, reporting and transmitting the data from the electromechanical drain to a wider ecosystem of interoperable components. In current art, there is no means to electronically establish or control drainage behaviors from a remote system and publish them to an electromechanical drain. Further, there is no way to establish normative values, including bolus, wean and titration limits on drains. Finally, the drain data today is manually charted in the patient record with a great degree of inaccuracy possible due to human error.

This process begins with the creation and approval process of the drain protocol safety library (DPSL). This DPSL accommodates the clinical behaviors regarding drainage protocol modalities, including lumbar and ventricular drains, where such protocol includes the primary identification of drain modality that then enables different clinical functions to be established and controlled on the device. The primary driver of protocol modality is the clinical decision to drain based on volume or pressure. The limits and behaviors are then categorized according to this gross function into protocols.

### SUMMARY OF THE INVENTION

The present disclosure presents apparatus, systems, and methods for fluid drain control, specifically of cerebrospinal fluid. The apparatus, systems, and methods present numerous improvements over the prior art, as described above.

In combination with the drain system, the state of the art can be extended to include rapid, recurrent measurement using fluid still in fluidic contact with the patient.

These aspects of the present invention, and others disclosed in the Detailed Description of the Drawings, represent improvements on the current art. This summary is provided to introduce a selection of concepts in a simplified form that are further described below in the Detailed Description of the Drawings. This Summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of various aspects, is better understood when read in conjunction with the appended drawings. For the purposes of illustration, the drawings show exemplary aspects; but the presently disclosed subject matter is not limited to the specific methods and instrumentalities disclosed. In the drawings, like reference characters generally refer to the same components or steps of the device throughout the different figures. In the following detailed description, various aspects of the present invention are described with reference to the following drawings, in which:

FIG. 1 shows schematic representations of typical human anatomy and placement and use of catheters.

FIG. 2 shows a schematic diagram of a system implementing an aspect of the present disclosure.

FIG. 3 shows a schematic diagram of an aspect of the present disclosure.

FIG. 4 shows a schematic diagram of an aspect of the present disclosure.

FIG. 5 shows a schematic diagram of an aspect of the present disclosure.

FIG. 6 shows a schematic diagram of an aspect of the present disclosure.

FIG. 7 shows a schematic diagram of an aspect of the present disclosure.

FIG. 8 shows a schematic diagram of an aspect of the present disclosure.

FIG. 9 shows a chart of a normal ICP wave form.

FIG. 10 shows a chart of an ICP waveform indicative of intracranial hypertension.

FIG. 11 shows charts of ICP waveforms classified as Lundberg A waves and Lundberg B waves.

FIG. 12 shows a schematic diagram of an aspect of the present disclosure.

FIG. 13 shows a schematic representation of a method of the present disclosure.

FIG. 14 shows a schematic representation of a method of the present disclosure.

FIG. 15 shows a schematic representation of a method of the present disclosure.

FIG. 16 shows a schematic representation of a method of the present disclosure.

FIG. 17 shows a schematic representation of a method of the present disclosure.

FIG. 18 shows a schematic representation of a method of the present disclosure.

FIG. 19 shows a schematic representation of a method of the present disclosure.

FIG. 20 shows a schematic representation of a method of the present disclosure.

FIG. 21 shows a schematic representation of a method of the present disclosure.

### DETAILED DESCRIPTION OF THE DRAWINGS

The presently disclosed invention is described with specificity to meet statutory requirements. But, the description itself is not intended to limit the scope of this patent. Rather, the claimed invention might also be configured in other ways, to include different steps or elements similar to the ones described in this document, in conjunction with other present or future technologies. Moreover, although the term “step” or similar terms may be used herein to connote different aspects of methods employed, the term should not be interpreted as implying any particular order among or between various steps herein disclosed unless and except when the order of individual steps is explicitly described. The word “approximately” as used herein means within 5% of a stated value, and for ranges as given, applies to both the start and end of the range of values given.

In the following description, numerous specific details are set forth to provide a thorough understanding of the invention. But, the present invention may be practiced without these specific details. Structures and techniques that would be known to one of ordinary skill in the art have not been shown in detail, in order not to obscure the invention. Referring to the figures, it is possible to see the various major elements constituting the apparatus, systems, and methods of use the present invention.

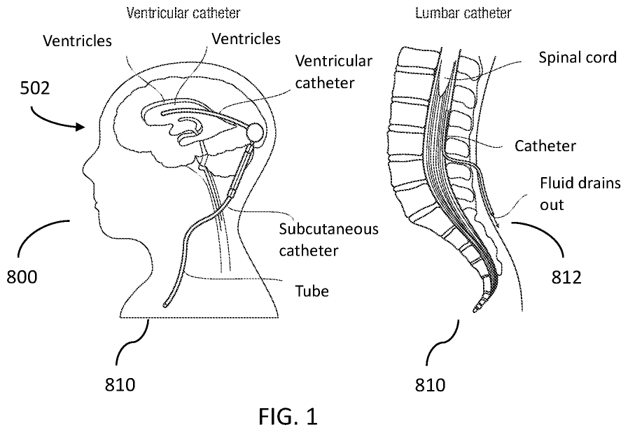

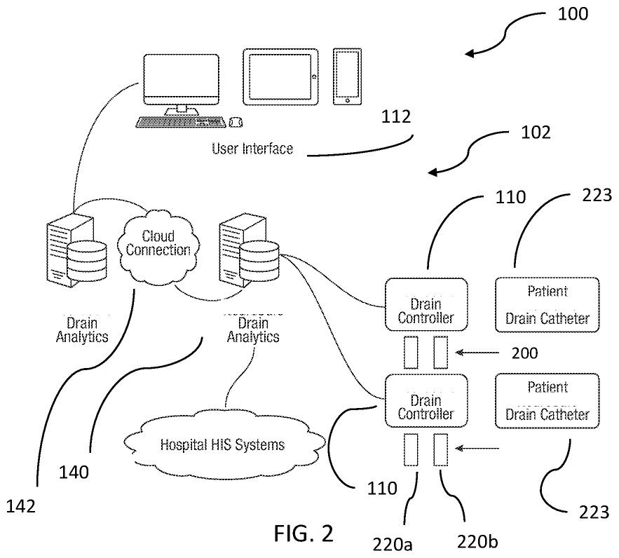

At a high level of abstraction, the fluid drain control apparatus, systems, and methods described herein operate or are operated in the environment depicted in FIG. 1, comprising a patient 800 having a body 810 and body fluids 812. The fluid drain control apparatus, systems, and methods comprise components as illustrated in FIG. 2-FIG. 8.

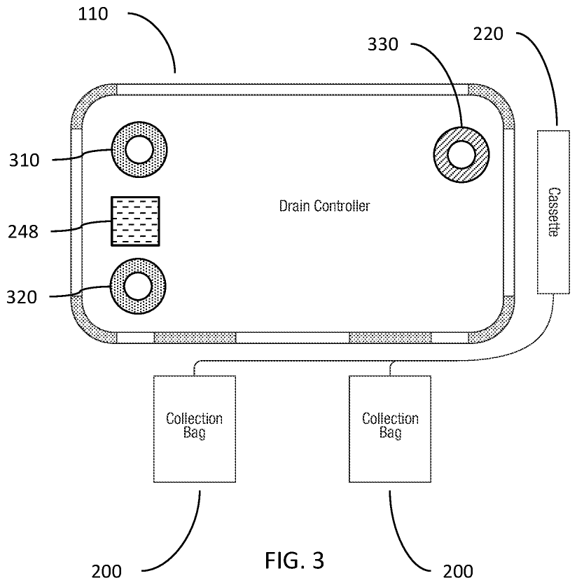

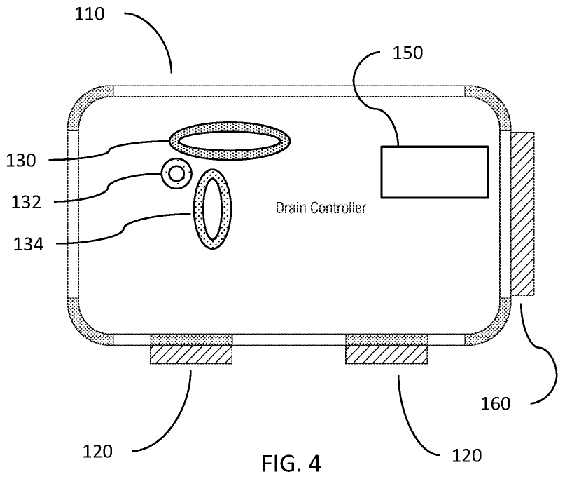

The system 100 for fluid drain control comprises a drain controller 110, which may be referred to herein as a “Drain Controller”. The system 100 further comprises a plurality of drainage collection bags 200, and a plurality of drain cassettes 220, as illustrated in FIG. 3 and FIG. 4. The drain controller 110 further comprises a plurality of drainage collection bag mounts 120. Each of the plurality of drainage collection bags 200 may be securely and reversibly affixed to and removed from any of the plurality of drainage collection bag mounts 120. The drain controller 110 further comprises a plurality of drain cassette mounts 160. Each of the plurality of drain cassettes 220 may be securely and reversibly affixed to and removed from any of the plurality of drain cassette mounts 160. Each of the plurality of drain cassettes 220 may be single use, referred to as single-use drain cassettes 220. Each of the plurality of drain cassettes 220 may comprise at least two planes. Each of the plurality of drain cassettes 220 may further comprise an elastomeric or silicone membrane compressed between at least two of the two planes. Any of the plurality of plurality of drain cassettes 220 may be designed for a specific therapy type, and in that aspect of the present disclosure, the drain controller 110 would automatically change to that type of therapy. For instance, and without limiting the foregoing, a drain cassette 220 that is for lumbar drainage may not have a transducer, as opposed to a drain cassette 220 that is for ventricular drainage would typically have a transducer—the drain controller 110 would prevent changing to an unsupported mode and would automatically select the correct mode for the therapy.

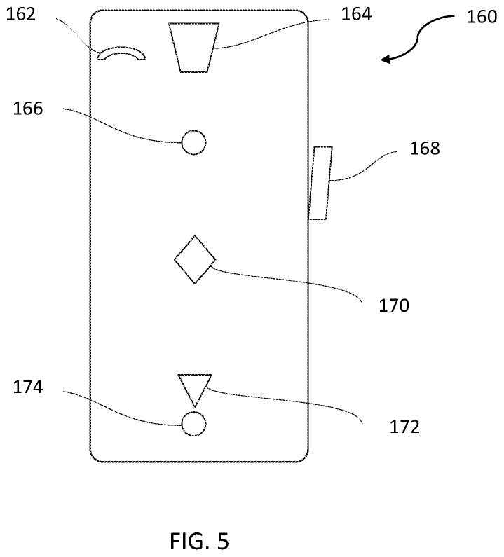

With reference to FIG. 5, each of the plurality of drain cassette mounts 160 further comprises a cassette identification reader 162, a pressure transducer interface 164, a valve actuator 166, a lever 168, a spectral analysis sensor 170, a fluid level sensor 172, and a valve actuator 174.

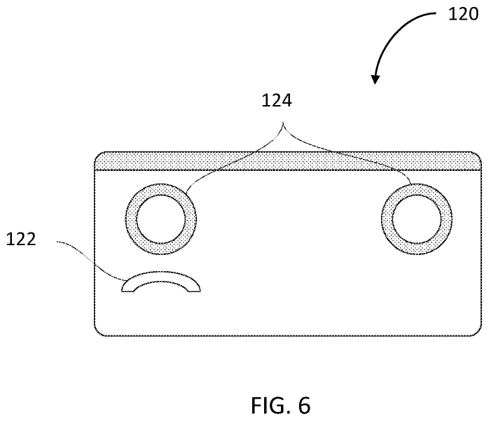

With reference to FIG. 6, each of the plurality of drainage collection bag mounts 120 further comprises components to enable and ensure safe management of accumulated bodily fluids, namely a collection bag identification reader 122, and a plurality of retention mechanisms and attached load cells 124.

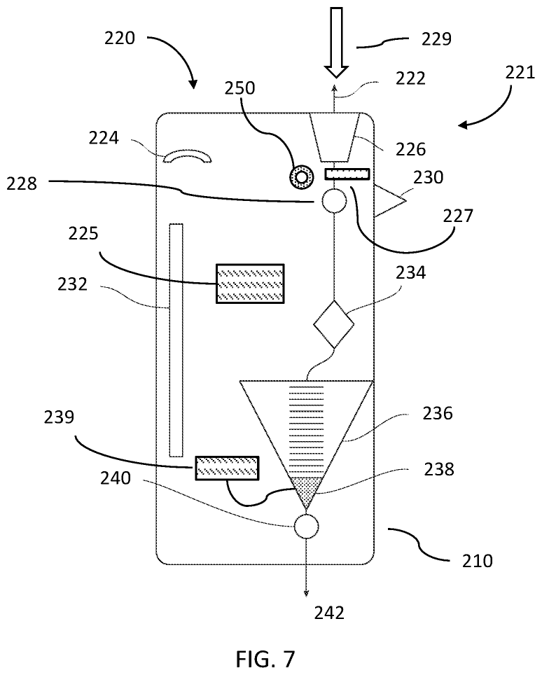

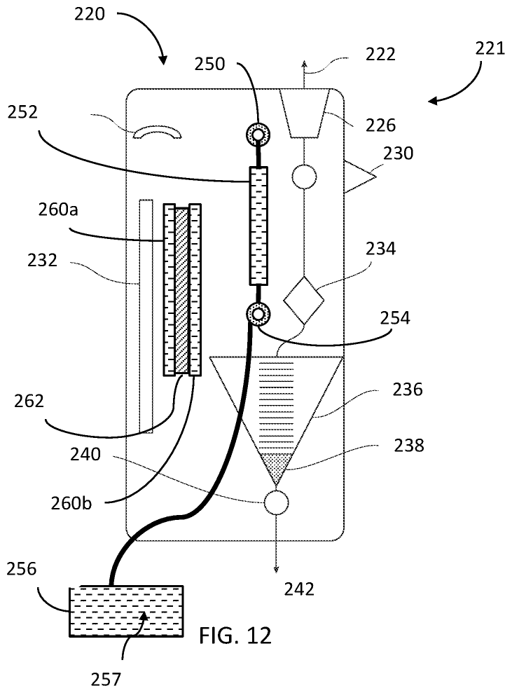

With reference to FIG. 7 and FIG. 12, each of the plurality of drain cassettes 220 further comprises components that facilitate the drainage of fluids from the body 810, namely a bag-catheter connection 222, a cassette identification 224, a pressure transducer 226, a multi-state valve 228, a sampling port 230, a finger-hold-pin 232, a spectral analysis port 234, a visual inspection port 236, a fluid sensor port 238, a two-way valve 240, and a bag-drain-collection connection 242.

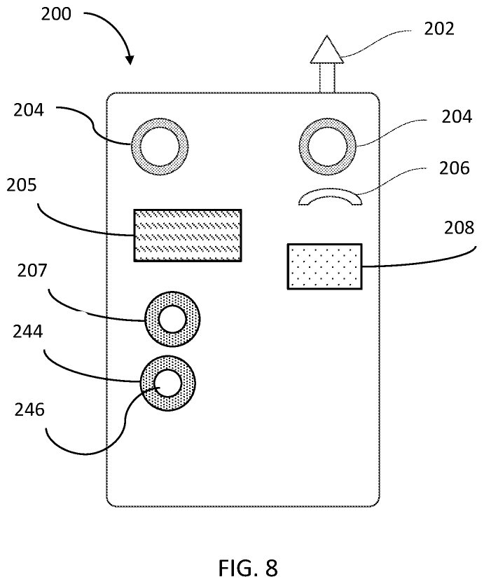

With reference to FIG. 8, each of the plurality of drainage collection bags 200 further comprises components to facilitate the collection of fluids drained from the body 810, namely a bag connection fitting 202, a plurality of bag-retention-supports 204, and a collection bag identification 206. The collection bag identification 206 may comprise, it has been found advantageous, a machine-readable identification mechanism that identifies at least one of: a manufacturer of the drainage collection bag 200, a date of manufacturing, an expiration date of the consumable, a maximum volume of the drainage collection bag 200, an identification of the drainage collection bag 200, a proper disposal information of the drainage collection bag 200, and a current amount of body fluids 812 in the drainage collection bag 200.

### Volumetric Drainage.

The fluid drain control apparatus, systems, and methods of the present disclosure further extend the known art to include limitable, titratable and bolus volumetric drainage systems with automated control of CSF samples, including concurrent sampling of CSF fluid and drainage, and including recording of the CSF sample size into the overall CSF volume drained during the period. The present disclosure further teaches including integration with off-the-shelf infusion pumps to perform lumbar infusion tests supporting normal pressure hydrocephals stress tests. It has been found advantageous to allow ICP sampling with active pressure compensation, as such sampling permits ICP drainage to be performed while actively monitoring a patient for ICP and/or draining CSF as needed from the patient; it will be understood that such sampling with active pressure compensation can be applied to any body fluids 812.

The present disclosure teaches an automated body fluid drain control system 102, the system comprising the drain controller 110; a plurality of drainage collection bags 200 wherein each of the drainage collection bags 200 has a variable size; and a multi-state valve 228 being a first controllable flow means having a variant number of states including but not limited to open to drain, partially open to drain, closed to drain, open to sample, partially open to sample, and closed to sample, such that it is possible for multiple states to be active concurrently within the multi-state valve 228. The automated body fluid drain control system 102 may be referred to as an electromechanical drain. The automated body fluid drain control system 102 may comprise a user interface 112. The automated body fluid drain control system 102 further comprises the fluid sensor port 238, being a measuring device that monitors the amount of fluid being drained. The automated body fluid drain control system 102 further comprises the two-way valve 240, wherein the two-way valve 240 is a second controllable flow means having an open and closed state.

In some aspects of the present disclosure, the fluid sensor port 238 of the automated body fluid drain control system 102 comprises or is connected to a fluid flow calculator 239, which fluid flow calculator 239 calculates a volumetric fluid flow 229 of CSF or other fluids on a periodic basis, the periodic basis being a time period or time scale appropriate for drainage of CSF or other fluids. The automated body fluid drain control system 102 adjusts the multi-state valve 228, the first controllable flow means, to reduce or increase the volumetric fluid flow 229 to fit uniformly within a calculated drainage volume desired for the time period appropriate for drainage of fluids. In some aspects, the multi-state valve 228 is connected to a patient 800 for draining body fluids 812 by gravity. In some aspects, the two-way valve 240 is connected to an output device, including but not limited to any of the plurality of drainage collection bags 200, for purposes of collecting the body fluids 812 which are being drained—the body fluids 812 may be referred to as “excess body fluids”. The body fluids 812 may comprise cerebrospinal fluid (CSF). In some aspects, the automated body fluid drain control system 102 further comprises a vent 244 that connects the drainage collection bags 200, i.e. the collection chamber, to open air, wherein the vent 244 comprises a filter 246, wherein the filter 246 may be of any of a range various sizes and filtration levels to prevent introduction of contaminants into the automated body fluid drain control system 102 and drainage collection bags 200, The body fluids 812 may be any fluid occurring in the body 810, produced in the body 810, or introduced into the body 810 (typically intentionally in medical practice, but could also include unintentional or accidental introduction of fluid into a body 810); such body fluids 812 may include but are not limited to cerebrospinal fluid; urine; a fluid pumped into the abdomen of a patient 800 and then drained, such as in peritoneal dialysis; or any other fluid now known or later invented.

In some aspects, the automated body fluid drain control system 102 further comprises a monitor system 248, wherein the monitor system 248 indicates an alarm when the body fluids 812 cannot or do not generate the volumetric fluid flow 229 to a flow volume that is requested or desired. The monitor system 248 can indicate an alarm when the automated body fluid drain control system 102 is not functioning. In some aspects, the automated body fluid drain control system 102 may comprise a spectral analysis port 234. In some aspects, the automated body fluid drain control system 102 may comprise a machine-readable identifier 235. In some aspects, the automated body fluid drain control system 102 may comprise a plurality of drain cassettes 220. Each or any of the plurality of drain cassettes 220 may be single-use or otherwise disposable. Each of the plurality of drain cassettes 220 may be used to isolate all biohazardous fluids from the automated body fluid drain control system 102. In some aspects, each of the plurality of drain cassettes 220 is operated with single-handed insertion into the automated body fluid drain control system 102, and/or single-handed removal from the automated body fluid drain control system 102.

### Continuous Pressure Monitoring Drainage.

The present disclosure teaches continuous intracranial pressure (ICP) monitoring of the CSF fluid that directly adjusts the drainage system to support ventricular CSF drainage. As discussed above, in existing drainage systems, the manual system of pressure monitoring involves manually opening and closing stopcocks and utilizing a combination of fluid pressure against head height to drain the patient to maintain a given intracranial pressure (ICP). These systems have an external fluid-filled transducer that measures the ICP of the patient via the pressure at the point of transducing. Alternatively, they may measure ICP via an implantable pressure sensor in the ventricular shunt. In either case, the pressure sensor is not in communication with the manual drain and provides no feedback to the user or control of the drainage amounts. Further, because the manual drains are not in communication with the pressure sensor, the accuracy of the pressure sensor varies depending on the unknown status of the stopcock. When open, the accuracy of the pressure sensor falls off and shows a significant reduction in pressure. When closed, the accuracy returns to nominal and the pressure values being monitored suddenly return to normal. A drain that is in communication with various means of monitoring ICP can thus adjust for the open and closed state of the drain to provide normalized pressure values for standardized pressure monitoring regardless of drain state. Further, unlike a manually operated drain, an automated system can open and close the drain multiple times per minute to both achieve the targeted ICP and provide highly accurate ICP monitoring of the patient. It has been found advantageous, in some aspects of the present disclosure, to have the system 100 and the methods of the present disclosure allow for passively monitoring ICP.

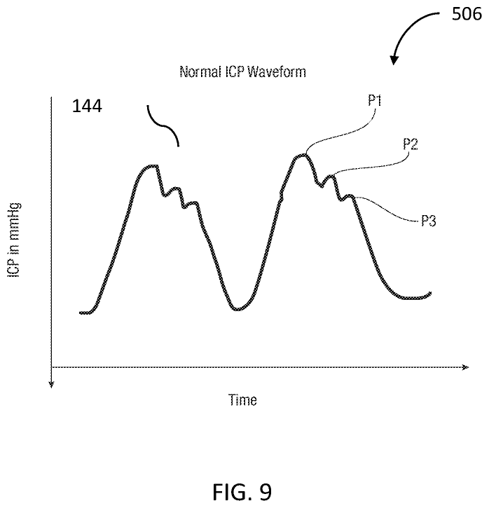

ICP waveforms can be characterized into normal and abnormal patterns. With reference to FIG. 9, a normal ICP wave form is illustrated, showing the P1 (Percussion wave), P2(Tidal wave), and P3 (Dicrotic notch), collectively referred to herein as normalized ICP waveform patterns 506.

Attempts have been made by Hammar et al to use the morphology of the ICP pulse wave as a surrogate marker of intracranial elastance. They decided that the systolic part of the vascular ICP waveform reflects arterial activity while the caudal descending segment denotes the pressure in SVC. Hence when the ICP increases the caudal part of the ICP waveform (the P2 component) assumes the shape of an arterial pulse and when there is CVP elevation, the waveform approximates a venous pulse.

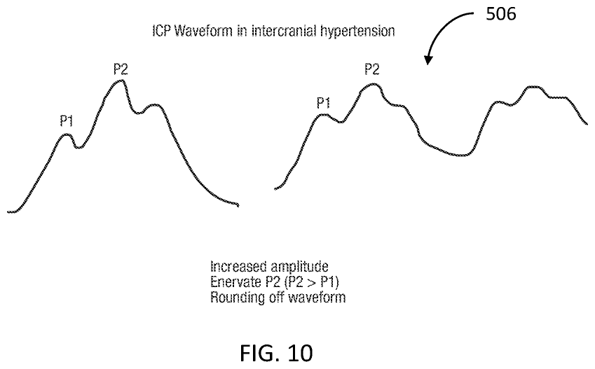

When the ICP is elevated, the vascular (cardiac) waveform amplitude increases while the respiratory waveform amplitude decreases. Other phenomena which are visible in dysfunctional intracranial compliance include occurrence of P waves as well as elevation of P2 and rounding off of the waveform. The occurrence of these phenomena are useful in clinical practice in that these alert the neurophysician to initiate ICP control measures on an urgent basis. It is pertinent to note here that increased ICP can produce characteristic waveform variously classified by Lundberg into A, B and C waves. The ICP waveform shown in FIG. 10 is indicative of intracranial hypertension.

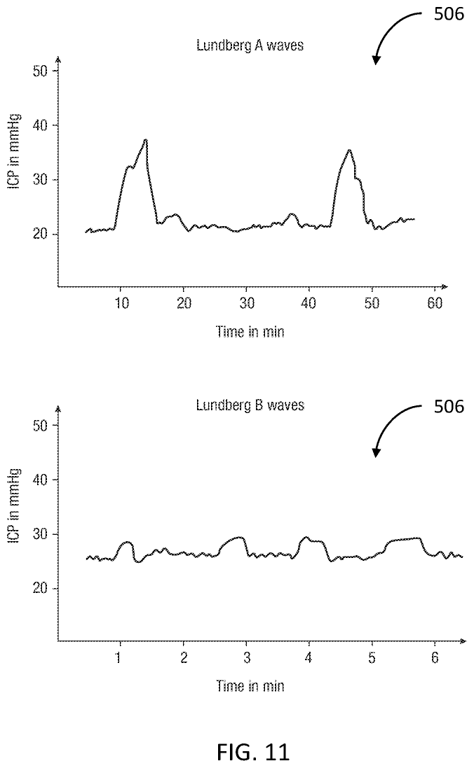

With reference to FIG. 11, Lundberg A waves are the ones which denote highest rise in ICP (50-100 mmHg). They are generally indicative of high degree of cerebral ischemic and impending brain herniation and persist for 5 to 10 min. Lunenburg B waves occur for a lesser period of time (1 to 2 min), the ICP elevation or not as much, 20 to 30 mm Hg, and are rhythmic in nature. They indicate evolving cerebral injury causing a gradual increase in ICP. Lundberg C waves correlate with blood pressure fluctuations brought about by baroreceptors and chemoreceptor reflex mechanisms and have no clinical significance.

An additional use case for CSF drainage is the treatment of normal pressure hydrocephalus (NPH). NPH is a clinical condition with enlarged intra-cerebral ventricles (Hakim & Adams, 1965), and symptoms of gait disturbance, enuresis and cognitive reduction (Fisher, 1982; Williams & Malm, 2016). The small step, shuffling gait is an early dominant symptom, which may be due to direct pressure on the midbrain gait center from an enlarged third ventricle (Lee, Yong, Ahn, & Huh, 2005). The gait disturbance offers an opportunity to evaluate the degree of disease progression (Chivukula et al., 2015; Williams et al., 2008), and may even help in prediction of good post-operative outcome (Gaff-Radford & Godersky, 1986).

When the underlying pathology is insufficient re-absorbtion of CSF surgical shunting of CSF to intravenous or peritoneal space may alleviate the symptoms, but the postoperative success depends on correct diagnosis. NPH coexists with, can be caused by, and may be mimicked by different forms of arteriosclerosis.

A direct diagnostic method for NPH is the constant infusion lumbar infusion test (LIT; Katzman & Hussey, 1970), where mock CSF is injected into the spinal cavity for passage through the Sylvian aqueduct intra-cranially in order to stress the CSF re-absorbtion ability (see Ryding, Kahlon, and Reinstrup, “Improved lumbar infusion test analysis for normal pressure hydrocephalus diagnosis”, Brain Behay. 2018 November; 8(11): e01125, last accessed 06/14/2021, at https://www.ncbi.nlm.nih.gov/pmc/articles/PMC6236248/).

The intent for LIT is that the lumbar infusion of mock CSF will increase the intracranial CSF volume. If the intra-cranial CSF volume increases, the only volume that can decrease in equal measure is the venous volume, since the intra-cranial tissues are incompressible. Likewise, the arterial blood volume delivered intra-cranially during each systole is compensated for by compression of the venous pool by the same amount. The increased venous outflow resistance due to the venous vascular compression causes an increase in intra-cranial pressure (ICP; Marmarou, Schulman, & Rosende, 1978).

The LIT test is a combination of volumetric lumbar infusion and ICP pressure monitoring. In one study, the lumbar infusion test was done using a constant infusion rate (0.80 ml/min) and regarded as positive if the steady state CSF plateau pressure reached levels of >22 mm Hg (resistance to outflow >14 mm Hg/ml/min).

Another diagnostic test for NPH is a CSF tap test (also known as: lumbar tap test, tap test, or Miller Fisher Test). This typically involves removing 30 mL of CSF through a lumbar puncture after which cognitive function is assessed.

NPH positive predictive values were 80% for lumbar infusion test and 94% for tap test. The system of the present disclosure supports both modes of drainage through its ability to interface externally with an infusion pump which is connected to a Y-site located distal to the patient but proximal to the system of the present disclosure. The infusion pump reports its infusion rates directly to the system of the present disclosure which monitors the ICP using its internal pressure transducer.

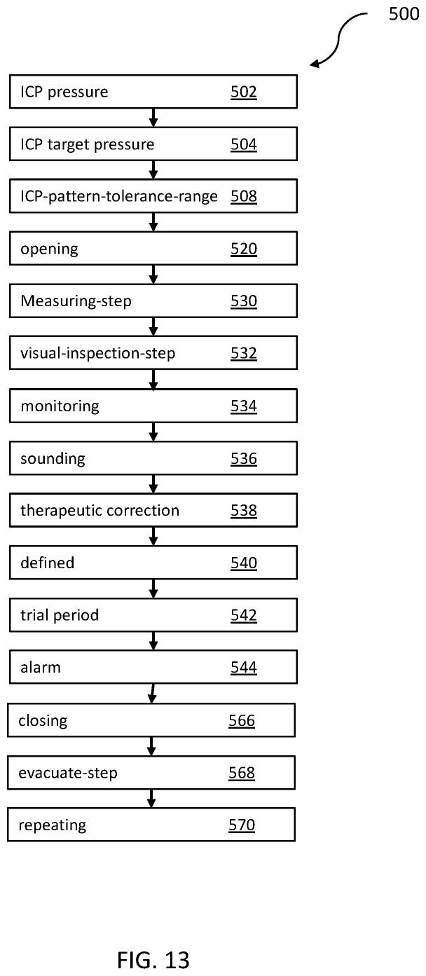

With reference to FIG. 13, the present disclosure teaches a method 500 for draining body fluids 812 from a body 810, wherein the method 500 is based on an ICP target pressure 504 that is desired, and wherein the method 500 is independent of volume of body fluids 812 drained. The method 500 comprises the steps of: measuring 510 an ICP pressure 502 through a connection to a pressure transducer 226, wherein the pressure transducer 226 may be a pressure monitor, sensor, or transducer, and wherein the pressure transducer 226 may be internal or external lo the body 810 or to an automated body fluid drain control system 102; if the ICP pressure 502 exceeds a ICP target pressure 504, opening 520 a drain 210 and allowing 522 an amount of body fluids 812 to drain through the natural pressure of the body 810 of the patient 800, or alternatively, allowing an amount of body fluids 812 to drain through a gravity-driven apparatus wherein body fluids 812 flow to the drain 210 positioned physically below the patient 800; a measuring-step 530 the amount of body fluids 812 drained; closing 566 the drain 210; and optionally, in a repeating 570 of the foregoing steps until the ICP target pressure 504 desire is achieved. The ICP pressure 502 may also be referred to herein as a plurality of inputs related to the ICP pressure 502, and/or as a plurality of values of the ICP pressure 502.

In some aspects of the present disclosure, the drain 210 comprises a plurality of drain cassettes 220, and wherein each of the plurality of drain cassettes 220 comprise a proximal valve 250, a graduated body 252, and a distal valve 254, and wherein the proximal valve 250 and the distal valve 254 connect to the graduated body 252. Each of the plurality of drain cassettes 220 may comprise a proximal portion 221, being the part of each of the plurality of drain cassettes 220 that is closer to the remainder of the automated body fluid drain control system 102, and the proximal valve 250 is located thereupon, and wherein the pressure transducer 226 is located near or above the proximal valve 250 in the proximal portion 221. The proximal portion 221 may further comprise a membrane 227 that can be interfaced to the pressure transducer 226 to measure the ICP pressure 502.

The proximal valve 250 may be a valve with two positions, or a valve with three positions, or a valve with multiple positions. The distal valve 254 may be a valve with two positions, or a valve with three positions, or a valve with multiple positions. In a valve with two positions, the valve has two possible positions, states, or conditions: open to drain, or closed. In a valve with three positions, the valve has three possible positions, states, or conditions: open to drain, open to sample, or closed. In a valve with multiple positions, the valve has multiple possible positions, states, or conditions, including but not limited to: variably open to drain and sample, fully open to drain, fully open to sample, or closed. In a valve with multiple positions, the drain controller 110 and/or the automated body fluid drain control system 102 may calculate an open, closed, or graduated position of the valve as the valve moves from 100% open to 100% sample to 100% closed, and in any of a range of intermediate states.

In some aspects of the present disclosure, opening 520 the drain 210 in the method 500 further comprises opening the proximal valve 250 and leaving the distal valve 254 closed to accumulate the body fluids 812 into the graduated body 252 of the plurality of drain cassettes 220 so that a user 890 may in a visual-inspection-step 532 visually inspect the volumetric fluid flow 229; the user 890 having a user profile, also referred to as a user class. The opening 520 the drain 210 may be done for a periodic amount of time, and that periodic amount of time may be variable. The maximum amount of body fluids 812 drained when the drain 210 is in the opening 520 state may be variable. In some aspects, the measured fluid volume that is drained correlates to the visual volume accumulated as noted in the visual-inspection-step 532 the volumetric fluid flow 229. In some aspects, the proximal valve 250 may be dosed and the distal valve 254 may be opened in an evacuate-step 568 the contents of the graduated body 252 into a disposal container 256. In some aspects, the volume 257 of body fluid 812 is recorded. The total volume of the disposal container 256 is known, and the volume 257 is compared in a total evacuated to the total volume of the disposal container 256, wherein the user 890 may be notified of required changes of the disposal container 256 as the disposal container 256 fills. In the foregoing methods and systems, the body fluids 812 may be CSF.

The automated body fluid drain control system 102 may further comprise an alignment element 130, which alignment element 130 may include but is not limited to a laser pointer affixed to at least one point on the automated body fluid drain control system 102, for aligning the automated body fluid drain control system 102 to the interventricular foramen of the patient 800 optically. In some aspects, the automated body fluid drain control system 102 further comprises a height-adjustment-control 132 for adjusting the height of the alignment element 130 without adjusting the physical height of the automated body fluid drain control system 102, wherein a height adjustment of the alignment element 130 is detected by the automated body fluid drain control system 102, and optionally, applying a known calculation adjustment to compensate for the overstatement or understatement of the ICP pressure 502 from at least one of the ICP pressure 502 inputs. In some aspects, the automated body fluid drain control system 102 further comprises means to adjust the physical height of the automated body fluid drain control system 102 through a mechanical, electromechanical, or manual adjustment. In some aspects, the automated body fluid drain control system 102 further comprises a patient-head-position-detection element 134, wherein the patient-head-position-detection element 134 may comprise a camera, a sensor, or other means of detecting the position of the head of the patient 800 and monitoring said position over time, such that the automated body fluid drain control system 102 can either automate an adjustment, or a prompt to the user 890 to adjust the position of the alignment element 130. In some aspects, the automated body fluid drain control system 102 allows the user 890 to manually apply a fixed adjustment to the ICP pressure 502 values to compensate for the overstatement or understatement of the ICP pressure 502. In some aspects, the automated body fluid drain control system 102 may automatically adjust between an ICP pressure 502 monitoring system that is interior to the ventricular system of the patient 800 and a pressure transducer 226 that is located exterior to the ventricular system of the patient 800.

In some aspects, the method 500 further comprises a monitoring 534 of the ICP pressure 502 for normalized ICP waveform patterns 506. When the ICP pressure 502 pattern does not meet the normalized ICP waveform patterns 506, the method 500 further comprises a sounding 536 of an alarm. In some aspects, the CSF drainage is applied as a therapeutic correction 538 to ICP pressure 502 patterns that are abnormal. The user 890 can, in some aspects of the method 500, set a defined 540 a trial period 542 for the body fluids 812 to correct any ICP pressure 502 pattern that is abnormal. In some aspects of the method 500, the automated body fluid drain control system 102 generates an alarm when the trial period 542 has ended if the ICP pressure 502 pattern remains abnormal, and/or the automated body fluid drain control system 102 generates an alarm 544 if the ICP pressure 502 pattern worsens, e.g. if the ICP pressure 502 pattern moves farther from the normalized ICP waveform patterns 506. In some aspects, the user 890 may select an ICP-pattern-tolerance-range 508, wherein the ICP-pattern-tolerance-range 508 is a range of patterns near or approximately near a normalized ICP waveform patterns 506 that is desired or normal, and wherein the user 890 may select the ICP-pattern-tolerance-range 508 during or after application of body fluids 812 drainage to correct the ICP pressure 502 pattern that observed by the user 890 or other observer. The alarm 544 may be altered or escalated by the method 500 if the ICP pressure 502 pattern exceeds the ICP-pattern-tolerance-range 508.

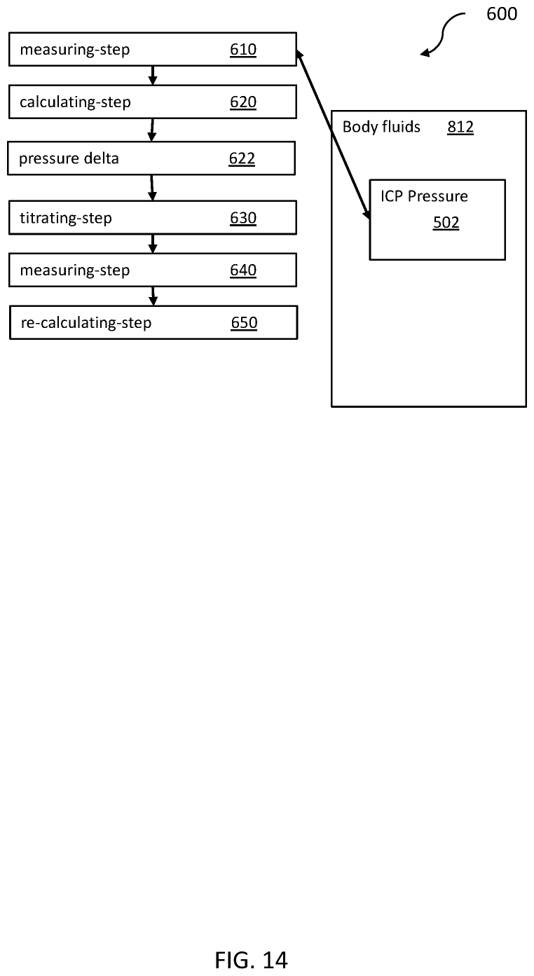

With reference to FIG. 14, the present disclosure teaches a method 600 for draining body fluids 812 from a body 810, wherein the method 600 is based on an ICP target pressure 504 that is desired, and wherein the method 600 is independent of volume of body fluids 812 drained. The method 600 comprises the steps of: a measuring-step 610 of an ICP pressure 502 through a connection to a pressure transducer 226, wherein the pressure transducer 226 may be a pressure monitor, sensor, or transducer, and wherein the pressure transducer 226 may be internal or external to the body 810 or to an automated body fluid drain control system 102. The method 600 further comprises a calculating-step 620 a pressure delta 622 by recording the ICP pressure 502 during a closed position of the proximal valve 250 and an open position of the proximal valve 250; then a titrating-step 630 the flow of the proximal valve 250 to achieve a ICP target pressure 504 that is desired; then a measuring-step 640 in which the method 600 measures the amount of body fluids 812 drained; and the method 600 further comprises a re-calculating-step 650 periodically the pressure delta 622 to ensure the pressure delta 622 remains within a range that is physiologically tolerable for the patient 800.

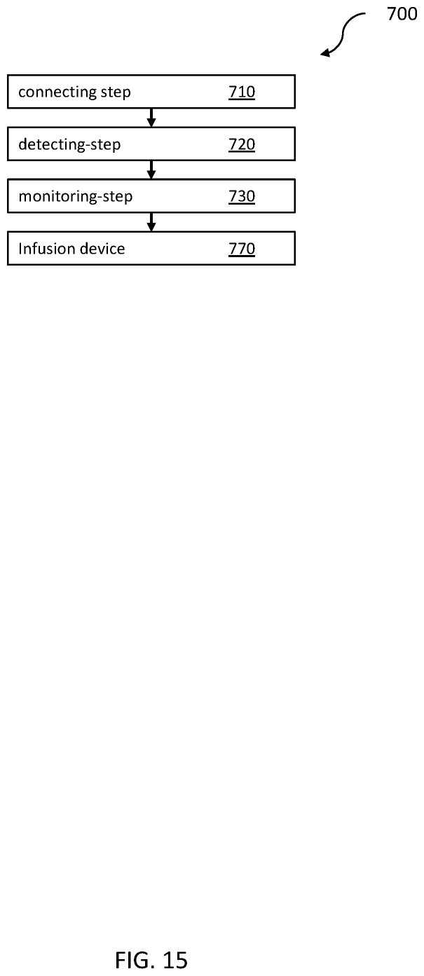

With reference to FIG. 15, the present disclosure teaches a method 700 for monitoring infusions of body fluids 812 into a catheter 223, wherein the catheter 223 is in fluidic connection with the automated body fluid drain control system 102, wherein the method 700 comprises the steps of: a connecting-step 710 an infusion device 770 to the automated body fluid drain control system 102, wherein the connecting-step 710 may be achieved physically, wirelessly, or through a network connection; a detecting-step 720 to detect the start and parameters, including but not limited to infusion rate, of the infusion device 770; and a monitoring-step 730 of monitoring the ICP pressure 502 through a connection to a multi-state valve 228 or a spectral analysis sensor 170. The method 700 may further comprise the automated body fluid drain control system 102 remotely-controlling 740 the infusion device 770, which remotely-controlling 740 may comprise starting infusions, stopping infusions, and the speed of infusions. The method 700 may further comprise the automated body fluid drain control system 102 controlling 750 a plurality of valve positions of the plurality of drain cassettes 220; and the method 700 may further comprise the user 890 manually controlling the plurality of valve positions of the plurality of drain cassettes 220.

### Drainage Cassette.

When operating under two different but similar drainage models, namely volumetric and pressure-oriented drainage as discussed herein, the automated body fluid drain control system 102 is improved in simplicity of use and practicality of use by a single-use consumable to facilitate correct operation. The single-use consumable must be simple to load into the automated body fluid drain control system 102, and simple to unload from the automated body fluid drain control system 102. This improvement, as taught by the present disclosure, is compounded by the need when using the systems and methods of the present disclosure, to identify the drainage model (volumetric or pressure-oriented) to apply to the patient 800 and to support spectrophotometric analysis of the body fluids 812 after the body fluids 812 have been drained. The teachings of both volumetric drainage models and pressure-oriented drainage models are thus extended to include the present disclosure of a single-use consumable that interfaces with the automated body fluid drain control system 102, in all models of usage and methods taught in the present disclosure.

In the present disclosure, each of the plurality of drain cassettes 220, which may be referred to as a first drain cassette 220a, a second drain cassette 220b, and so on for any number in the plurality of drain cassettes 220, comprises at least a first planar element 260a and at least a second planar element 260b. The first drain cassette 220a may be a single-use cassette, as may be the other cassettes in the plurality of drain cassettes 220. In each of the plurality of drain cassettes 220, the first drain cassette 220a may further comprise a membrane 262, wherein the membrane 262 is compressed between the first planar element 260a and the second planar element 260b, and wherein the membrane 262 may be elastomeric, or silicone, or other material now known or later invented. In some aspects, the first drain cassette 220a, the second drain cassette 220b, and any other cassettes in the plurality of drain cassettes 220 may comprise a proximal valve 250, wherein the proximal valve 250 is in fluidic contact with body fluids 812 of a patient 800 and the body fluids 812 require drainage; and the plurality of drain cassettes 220 may comprise a distal valve 254, wherein the distal valve 254 is in fluidic contact with one or more of a plurality of drainage collection bags 200 that will collect the body fluids 812; and wherein the proximal valve 250 and the distal valve 254 are in fluidic contact with each other; and wherein each of the proximal valve 250 and the distal valve 254 are capable of at least two of the following states: open, closed, and partially open for reduced flow.

In some aspects, the first drain cassette 220a, the second drain cassette 220b, and any other cassettes in the plurality of drain cassettes 220 may comprise a sampling port 230, wherein the sampling port 230 is in fluidic contact with the proximal valve 250, and wherein the sampling port 230 is either an open luer or a closed luer access point, including but not limited to needle-less access valves, pre-pierced ports, or other means of fluidic access. In some aspects, the first drain cassette 220a, the second drain cassette 220b, and any other cassettes in the plurality of drain cassettes 220 may comprise a visual inspection port 236 suitable for visual inspection of the body fluids 812. In some aspects, the first drain cassette 220a, the second drain cassette 220b, and any other cassettes in the plurality of drain cassettes 220 may comprise a fluid sensor port 238 that measures the amount of body fluids 812 that was or has been drained from the patient 800. In some aspects, the first drain cassette 220a, the second drain cassette 220b, and any other cassettes in the plurality of drain cassettes 220 may comprise a spectral analysis port 234, wherein the spectral analysis port 234 is suitable for spectrophotometry or other analytic sensor types to analyze the body fluids 812 prior to entry into the drainage collection bags 200 or other suitable drainage collection chamber. In some aspects, the first drain cassette 220a, the second drain cassette 220b, and any other cassettes in the plurality of drain cassettes 220 may comprise a pressure transducer 226, wherein the pressure transducer 226 is in fluidic contact with the body fluids 812 prior to the proximal valve 250. The proximal valve 250 may be the same component as the multi-state valve 228, or the proximal valve 250 may be a different component from the multi-state valve 228.

In some aspects, the first drain cassette 220a, the second drain cassette 220b, and any other cassettes in the plurality of drain cassettes 220 may comprise a cassette identification 224, wherein the cassette identification 224 may, it has been found advantageous, be a machine-readable identification component that identifies at least item from the following list: a cassette manufacturer; a date of manufacturing; an expiration date of the consumable; a set of drainage modes supported by the cassette; an identification of the cassette; a proper disposal information of the cassette; a presence of various single-use cassette features, including but not limited to a known length of the proximal fluidic circuit and a known length of the distal fluidic circuit; a type of sampling port present; if alarm light indicators are present; a collection chamber size; and a type of analysis supported. In some aspects, the first drain cassette 220a, the second drain cassette 220b, and any other cassettes in the plurality of drain cassettes 220 may comprise a machine-writable identification mechanism 225, wherein the machine-writable identification mechanism 225 enables the apparatus to write a plurality of utilization and programming information into the first drain cassette 220a or other cassette such that, if the first drain cassette 220a is removed from the automated body fluid drain control system 102 and placed in a different automated body fluid drain control system 102, the plurality of utilization and programming information can be read by that different automated body fluid drain control system 102. The machine-writable identification mechanism 225 provides additional benefits in that it can help prevent counterfeiting, and can help prevent medical errors regarding which bag is used and associated with a patient.

### Drainage Bag.

For safe handling, any body fluids 812 must be stored in a sealed environment that prevents contaminants from entering the system, and the body fluids 812 must not be allowed to leak from the system, contaminating the greater environment that the medical facility operates in, from or with biohazardous material. The automated body fluid drain control system 102 of the present disclosure is thus extended to include a plurality of drainage collection bags 200. It has been found advantageous to have the plurality of drainage collection bags 200 each be single-use, disposable, and secure. Each of the plurality of drainage collection bags 200 comprises a flexible container for containment of biohazardous or potentially biohazardous body fluids 812, wherein each of the plurality of drainage collection bags 200 comprises at least 250 mL of fluid storage, and comprises at least one fitting 202 for fluid ingress, and comprises a plurality of bag-retention-supports 204, wherein each of the plurality of bag-retention-supports 204 is suitable to hold the drainage collection bags 200 if the drainage collection bags 200 were completely filled with body fluids 812. In some aspects of the present disclosure, the fitting 202 is a tubing connection with a male connector suitable for connection to a drainage system output port, luer, or other fitting now known or later invented; in other aspects of the present disclosure, the fitting 202 is a tubing connection with a female connector suitable for connection to a drainage system, and may present with a needle-less access valve,

In some aspects of the present disclosure, the drainage collection bags 200 further comprise a filtered vent 205. In some aspects of the present disclosure, the fitting 202 further comprises a check valve 207. In some aspects of the present disclosure, the drainage collection bags 200 further comprise a collection bag identification 206 mechanism, wherein the collection bag identification 206 is a machine readable mechanism that identifies at least one of the following list: the manufacturer of the drainage collection bags 200; the date of manufacturing; the expiration date of the drainage collection bags 200; the maximum volume of the drainage collection bags 200; the identification of the drainage collection bags 200; the proper disposal information of the drainage collection bags 200; and the current amount of fluid in the drainage collection bags 200. In some aspects of the present disclosure, the drainage collection bags 200 may further comprise a machine-writable identification mechanism 208, wherein the machine-writable identification mechanism 208 enables the automated body fluid drain control system 102 to write a plurality of utilization and programming information into the drainage collection bags 200 such that, if the drainage collection bags 200 is removed from the automated body fluid drain control system 102 and placed in a different automated body fluid drain control system 102, the plurality of utilization and programming information can be read by that different automated body fluid drain control system 102. The collection bag identification 206 provides additional benefits in that it can help with safety checks, such as the amount of fluid in the drainage collection bag 200, and can help prevent medical errors regarding which drainage collection bag 200 is used and associated with a patient. The collection bag identification 206 may also help prevent medical errors by precluding any reuse of a drainage collection bag 200. The collection bag identification 206 and/or the machine-writable identification mechanism 208 may comprise a RAD (radio-frequency identification) chip and reader, optical code and reader, or other mechanism now known or later invented.

### Drain Controller.

In the present disclosure, the foregoing disclosures can be combined and made or used in any combination, including but not limited to combining and using the disclosures of the Volumetric Drainage and Continuous Pressure Monitoring Drainage, the Drainage Cassette, and the Drainage Bag, together being an aspect of the system of the present disclosure. The automated body fluid drain control system 102 may partially or completely automate the process of draining body fluids 812, cerebrospinal or other, from a patient 800. In some aspects, the automated body fluid drain control system 102 further comprises input ports 310, wherein the input ports 310 may be used for external connections, including but not limited to ICP, ART, and Flushless Tranducers. In some aspects, the automated body fluid drain control system 102 further comprises output ports 320, wherein the output ports 320 may be used for patient monitors, including but not limited to bedside monitors. The automated body fluid drain control system 102 may further comprise a plurality of alarm indicators 330, wherein the alarm indicators 330 may be located on at least one point on the automated body fluid drain control system 102, and wherein the alarm indicators 330 may flash, change color, or otherwise alert a user 890 that an alarm state exists and thus that action may be required. In some aspects, the automated body fluid drain control system 102 may detect whether or not the plurality of drain cassettes 220 is inserted correctly or not; has expired; is not authentic; needs to be primed; or other conditions that may be helpful to the user 890. In some aspects of the present disclosure, when a cassette from a plurality of drain cassettes 220 is inserted, the automated body fluid drain control system 102 may change to the correct drainage mode if the plurality of drain cassettes 220 so indicates. In some aspects of the present disclosure, when no cassette from a plurality of drain cassettes 220 is inserted, the automated body fluid drain control system 102 may display on a user interface 112 information, including but not limited to animations, text, or schematics, showing how to load a first drain cassette 220a or other cassette from the plurality of drain cassettes 220. In some aspects of the present disclosure, the automated body fluid drain control system 102 may prompt a user 890 to change the drainage collection bags 200, or to prompt that no drainage collection bags 200 are attached.

In some aspects of the present disclosure, when one of the plurality of drainage collection bags 200 is attached, the automated body fluid drain control system 102 will advantageously: display, on the user interface 112, the amount of fluid in the drainage collection bags 200; warn the user 890 when the automated body fluid drain control system 102 detects that the drainage collection bags 200 has, or have, in them a user-configurable percentage of body fluids 812 collected, as a percentage of their total possible storage of body fluids 812; warn the user 890 if the drainage collection bags 200 has expired; and/or warn the user 890 if the bag is not authentic. In some aspects of the present disclosure, when no drainage collection bag from a plurality of drainage collection bags 200 is inserted, the automated body fluid drain control system 102 may display on a user interface 112 information, including but not limited to animations, text, or schematics, showing how to load one or more drainage collection bags 200. The user interface 112 may be a wireless or wired interface, and may be comprise a touchscreen interface, gestural controls, or other input mechanisms now known or later invented. The user interface 112 may comprise a machine-readable user identification system, including but not limited to user tags, RFID cards, biometric data, passwords, user names, and/or user proximity). The automated body fluid drain control system 102 may enable, based upon the authentication of a user 890 at the user interface 112, a set of privileges, including but not limited to an ability to change the program on the automated body fluid drain control system 102.

### Spectrophotometric Analysis of Cerebrospinal Fluid Over Time.

Traditionally in the prior art, cerebrospinal fluid (CSF) has been examined using a process that pulls a sample from of CSF from a patient based on an identified risk or event. The fluid is then examined in a laboratory setting to detect blood and blood products from haemorrhage. Fluid from patients with this condition will contain red blood cells unless they have been completely metabolized—an event which typically takes at least 7 days to occur. Red blood cells lyse, releasing oxyhaemoglobin which is then converted into bilirubin. After centrifugation, the CSF supernatant is visible pink or pink-orange in color from oxyhaemoglobin, yellow due to billirubin and intermediate if both are present.

With the advent of spectrophotometry, the laboratory is now able to identify data without the introduction of centrifuges and other laborious processes. It is common to identify oxyhaemoglobin (413-415 nm), oxyhaemoglobin and bilirubin (broad peak/shoulder at 450-460 nm), and bilrubin alone. Methaemlobin may also be identified (405 nm shifting to 413 nm when oxyhaemgloin is present). It is also possible to identify glucose (˜1500 nm) and insulin (˜260-350 nm) in bodily fluids and proteins at ˜1575 nm.

All of these methods rely on traditional laboratory techniques and instruments. The application occurs against the entire column of fluid and requires re-sampling each time the test needs to be run. In combination with the drain system, the state of the art can be extended to include rapid, recurrent measurement using fluid still in fluidic contact with the patient.

In some aspects of the present disclosure, the automated body fluid drain control system 102 comprises a plurality of spectrophotometric sensors and a plurality of light sources, wherein the plurality of light sources may be able to emit light and the plurality of spectrophotometric sensors may be able to sense light, in a range of wavelengths ranging from approximately 250 nm-approximately 1900 nm. The plurality of spectrophotometric sensors and light sources may be comprised separately, or may comprise the spectral analysis port 234 of the automated body fluid drain control system 102. The analysis of the body fluids 812 by the automated body fluid drain control system 102 may, it has been found advantageous, be applied not against a column of idle collected body fluids 812, but rather against the actively draining fluid, both in minute quantities (advantageously, <0.5 mL) and hi very small time increments (advantageously, less than 1 minute), In some aspects of the present disclosure, the foregoing data processing, analysis, and storage occur on the automated body fluid drain control system 102, such that, without limitation, each spectrophotometric signature can be performed, analyzed and stored on the automated body fluid drain control system 102 even when not in contact with a data platform. The foregoing data processing, analysis, and storage may further comprise including data indexing against identification of the plurality of drain cassettes 220, drainage session, and/or information on the patient 800. The automated body fluid drain control system 102 may further comprise an online data platform for the foregoing data processing, analysis, and storage, referred to as a data platform 140, which may be referred to herein as the “data platform”. In some aspects, the automated body fluid drain control system 102 may be able to receive a plurality of signatures 142 which may be stored for upload to the data platform 140. The automated body fluid drain control system 102 may be able to store normative signature-patterns 144, and store them on the automated body fluid drain control system 102. The automated body fluid drain control system 102 may be able to download normative signature-patterns 144 to be stored on the automated body fluid drain control system 102. The automated body fluid drain control system 102 may be able to alert a user 890 with an alarm if one of the plurality of signatures 142 deviates from the normative signature-patterns 144. In some aspects of the present disclosure, the automated body fluid drain control system 102 is able to conduct an infusion test, wherein a known fluid is injected into the CSF space (traditionally lumbar), the known fluid is monitored and analyzed for changes in drainage fluid spectrographic signature to determine amount of dilution, if any, and when and after what volume of drained cerebrospinal fluid has the cerebrospinal fluid returned to a normal level, which may also be referred to as a pre-infusion level or a pre-infusion-test or a pre-infusion-test level.

### Drain Safety System, Protocols and Analytics.

With the advent of electromechanical fluid drains, it now becomes possible to develop and implement clinically driven safety protocols that control and instruct the device to more safely drain body fluids from a given patient population. In today's environment, all patients are treated as identical by the device. With this enhancement to the art, the device will become aware of the unique physiological parameters of the patient and the clinical diagnosis', including comorbidities, which will instruct the device in how to properly monitor and drain the patient. Further, the same device can be customized to any given patient through the use of a set of clinical parameters that are entered by the clinician on the device to select the unique drain conditions that are applicable to this patient.

We also consider user preference and needs for novel methods of entering, selecting, reporting, and transmitting the data from the electromechanical drain to a wider ecosystem of interoperable components. In current art, there is no means to electronically establish or control drainage behaviors from a remote system and publish them to an electromechanical drain. Further, there is no way to establish normative values, including bolus, wean and titration limits on drains. Finally, the drain data today is manually charted in the patient record with a great degree of inaccuracy possible due to human error.

This process begins with the creation and approval process of the drain protocol safety library 900 (DPSL). This drain protocol safety library 900 accommodates the clinical behaviors regarding drainage protocol modalities, including lumbar and ventricular drains, where such protocol includes the primary identification of drain modality that then enables different clinical functions to be established and controlled on the device. The primary driver of protocol modality is the clinical decision to drain based on volume or pressure. The limits and behaviors are then categorized according to this gross function into protocols.

In the following desirable or advantageous functions, various modes and functions are set forth. If the desirable or advantageous functions are not met in a mode of operation of the systems and methods of the present disclosure, the systems and methods can be and advantageously are set to trigger alarms or alerts. In one aspect of the automated body fluid drain control system 102, operating in a volume-oriented drain modality, typical, but not exclusive, functions may include but are not limited to:
a Volumetric Metric drainage amount over some period of time
b Volo Metric drainage cycle time of valve control over a shorter period of time than 1.a
c Bolus drainage amount over some period of time
d Maximum volumetric drainage amount over some period of time
e Total time to drain

In one aspect of the automated body fluid drain control system 102, operating in a plurality of pressure-oriented drain modalities, typical, but not exclusive, functions may include but are not limited to:
a Maximum pressure at which to drain
b Cycle time of valves to control and monitor 2.a
c Maximum volumetric drainage amount over some period of time
d Total time to drain

The foregoing basic functions can then have improved safety parameters applied to them by creating a series of protocols that are first differentiated by modality, then by the patient population characteristics, including mnemonic representations for ease of identification, checklists of items the clinician must setup to initiate the protocol, and finally limits for each concept included in the function such that any one function can be protected. As an example, without excluding other possible limits, these limits could include:
a Default value to be used
b Whether the default value can be edit by the clinician at the bedside
c Maximum upper value the clinician cannot go beyond and the message to be displayed if attempted
d Recommended upper value the clinician may go beyond but will be warned is abnormal and the message to be displayed if attempted
e Recommended lower value the clinician may go beyond but will be warned is abnormal and the message to be displayed if attempted
f Minimum lower value the clinician cannot go beyond and the message to be displayed if attempted
g Maximum percentile change per titration event and the message that will be displayed if attempting to go beyond this limit (for instance, a drain at 10 mL/hour with a 10% titration would only allow a maximum change of 1 mL per titration event)
h Minimum percentile change per titration event and the message that will be displayed if attempting to go beyond this limit
i Definition of titration lockout period that the percentile change limits apply to (for instance, a drain at 10 mL/hour with a 10% titration limit and a lockout of 5 minutes would prevent a user from changing in 1 mL increments more frequently than every 5 minutes)

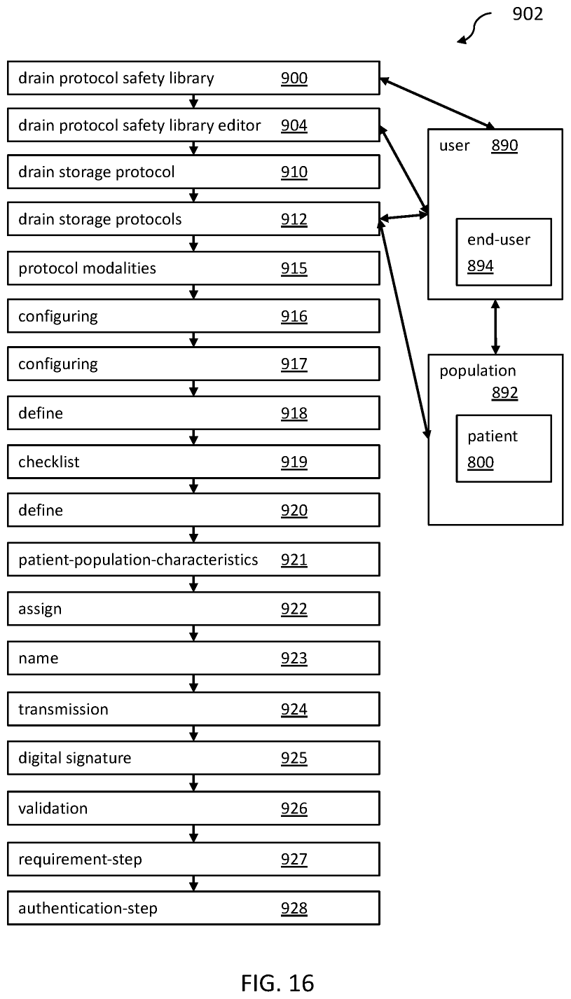

In one aspect, and with reference to FIG. 16, the present disclosure teaches an automated body fluid drain control system 102 that is capable of receiving, storing, selecting, executing, and operating on a method 902 for a drain storage protocol 910, wherein the drain storage protocol 910 may be designed for volumetric drainage or the drain storage protocol 910 may be designed for pressure-based drainage. The method 902 may further comprise the ability to design and implement a plurality of drain storage protocols 912 that allow clinicians, wherein clinicians may be the user 890, to develop drainage safety based on the needs of a population 892 of the patients 800, and wherein the method 902 further comprises having the automated body fluid drain control system 102 implement the drain storage protocols 912 to safely drain body fluids 812 from the body 810. In such aspects, the method 902 comprises the steps of: enabling a user 890 to select 914 one of a plurality of protocol modalities 915, the plurality of protocol modalities 915 including but not limited to lumbar and ventricular drainage; and enabling the user 890 to use a configuring 916 function to customize the behaviors and/or functions of the automated body fluid drain control system 102. The method 902 may comprise a configuring 917 of a plurality of drain storage protocol 910 and/or a plurality of drain storage protocols 912 into a drain protocol safety library 900. In some aspects of the method 902, the user 890 is required to approve the drain storage protocol 910, which may be done with a plurality of credentials of the user 890, to authenticate the drain storage protocol 910 as valid prior to utilization of the drain storage protocol 910. In some aspects of the method 902, the user 890 is required to approve the drain protocol safety library 900 as valid prior to utilization of the drain protocol safety library 900. The user 890 may use a define 918 step for a checklist 919 of steps the user 890 must perform prior to engaging the drain storage protocol 910 and/or the drain protocol safety library 900. The user 890 may use a define 920 step for a plurality of patient-population-characteristics 921 that apply to the drain storage protocol 910 and/or the drain protocol safety library 900. The user 890 may carry out an assign 922 step of a name 923 to drain storage protocol 910, as a reminder to a user 890, who may be a different person, of what the protocol function or purpose is, including but not limited to an end-user 894 of the automated body fluid drain control system 102, wherein the end-user 894 may need to have any of a set of characteristics or authorizations to utilize a drain storage protocol 910. The drain storage protocol 910 can be sent in a transmission 924 electronically to the automated body fluid drain control system 102, and in some aspects, the drain storage protocol 910 may be given a digital signature 925 prior to transmission 924, and the digital signature 925 receives a validation 926 by the automated body fluid drain control system 102 prior to acceptance of the drain storage protocol 910. In some aspects of the method 902, the automated body fluid drain control system 102 comprises a requirement-step 927 that the user 890 must act in an authentication-step 928 into the automated body fluid drain control system 102, and the authentication-step 928 is used by the method 902 to determine which of the plurality of drain storage protocols 912 the user 890 is authorized to utilize.

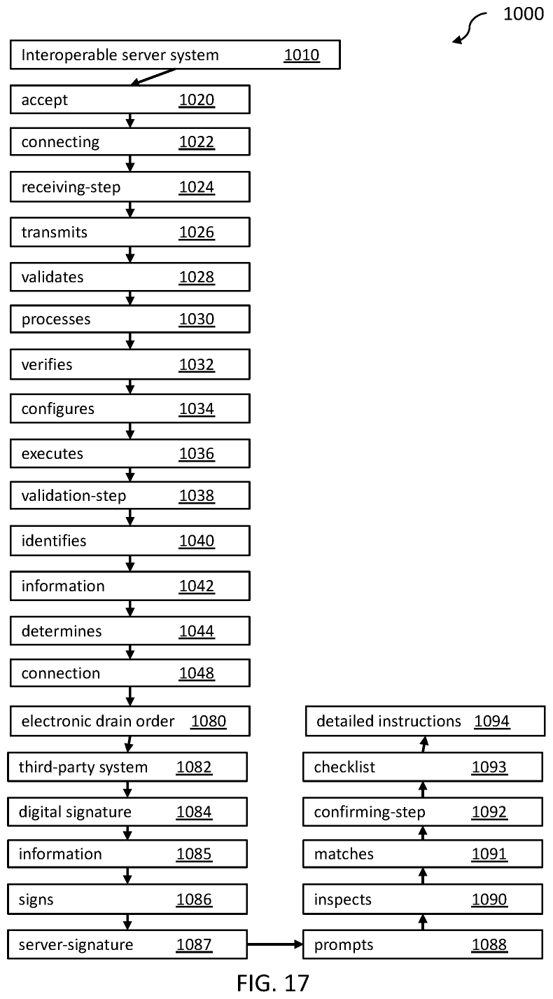

In one aspect, and with reference to FIG. 17, the present disclosure teaches a method 1000 for accepting an electronic drain order 1080 from a third-party system 1082, and transmitting it to an automated body fluid drain control system 102, the method comprising an interoperable server system 1010 that can carry out an accept 1020 step of the electronic drain order 1080 from the third-party system 1082, the method connecting 1022 to the automated body fluid drain control system 102; and after the interoperable server system 1010 receives, in a receiving-step 1024, an electronic drain order 1080 from the third-party system 1082, the interoperable server system 1010 transmits 1026 the electronic drain order 1080 to the automated body fluid drain control system 102. Thereafter, the automated body fluid drain control system 102 validates 1028 that the electronic drain order 1080 came from an interoperable server system 1010 that had been validated by the method 1000, and the automated body fluid drain control system 102 processes 1030 the electronic drain order 1080, the automated body fluid drain control system 102 verifies 1032 that the electronic drain order 1080 is within a plurality of physical parameters of the automated body fluid drain control system 102 to perform, the electronic drain order 1080 is used in a step to configures 1034 the automated body fluid drain control system 102, and the automated body fluid drain control system 102 executes 1036 the electronic drain order 1080. The electronic drain order 1080 may be to start a new drain order, or may be a titration of an existing drain order, or may be a stop of an existing drain order, or may be a pause of an existing drain order.

In some aspects, the third-party system 1082 may be validated, in a validation-step 1038, as a third-party system 1082 that has been approved. In some aspects, the electronic drain order 1080 identifies 1040 a particular automated body fluid drain control system 102 to which the electronic drain order 1080 is to be sent. In some aspects of the present disclosure, the interoperable server system 1010 has or is given a plurality of information 1042 that indicates the particular automated body fluid drain control system 102 to which the electronic drain order 1080 is to be sent, and the interoperable server system 1010 determines 1044 if the interoperable server system 1010 has a current and valid connection to that particular automated body fluid drain control system 102. In some aspects, the interoperable server system 1010 attempts to initiate 1046 a connection 1048 to the automated body fluid drain control system 102 if the connection 1048 does not exist. In some aspects, the interoperable server system 1010 inspects a digital signature 1084 of the electronic drain order 1080, prior to the interoperable server system 1010 accepting the electronic drain order 1080 as valid, wherein the digital signature 1084 contains an information 1085 for securing or verifying the contents and provenance of the electronic: drain order 1080. In some aspects, the interoperable server system 1010 signs 1086 the contents of the electronic drain order 1080 with a server-signature 1087 prior to submitting the electronic drain order 1080 to the automated body fluid drain control system 102. The automated body fluid drain control system 102 may inspect the server-signature 1087 and/or the digital signature 1084 prior to accepting the electronic drain order 1080 as valid. In some aspects, the automated body fluid drain control system 102 prompts 1088 the user 890 to accept the electronic drain order 1080 as valid before the automated body fluid drain control system 102 executes 1036 the electronic drain order 1080. In some aspects, the automated body fluid drain control system 102 inspects 1090 the electronic drain order 1080 and matches 1091 the electronic drain order 1080 to a drain storage protocol 910 that is valid and in the drain protocol safety library 900. In some aspects, the automated body fluid drain control system 102 may confirm, in a confirming-step 1092, that is, require a match of, the electronic drain order 1080 to a drain storage protocol 910 that is valid and in the drain protocol safety library 900, and the automated body fluid drain control system 102 rejects any electronic drain order 1080 that does not match a drain storage protocol 910 that is valid and in the drain protocol safety library 900. In some aspects, the drain storage protocol 910 contains a checklist 1093 that is presented to the user 890 as steps to be completed prior to starting the drain storage protocol 910, and the checklist 1093 may comprise detailed instructions 1094 comprising text, images, video, or other material for the user 890 to review before performing the steps of the checklist 1093.

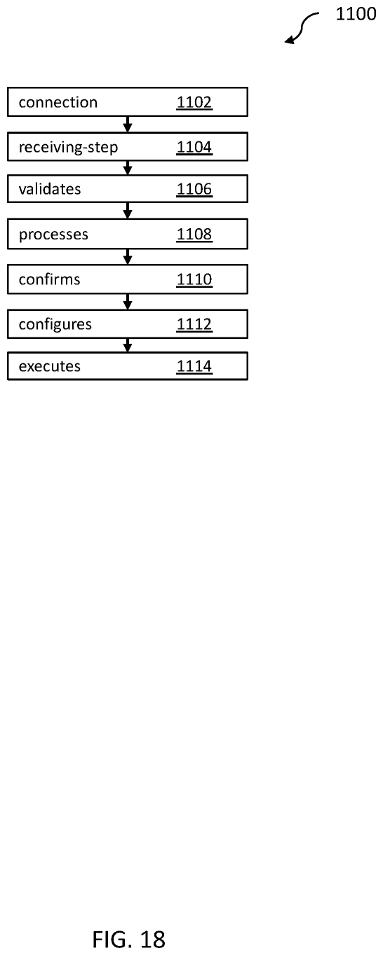

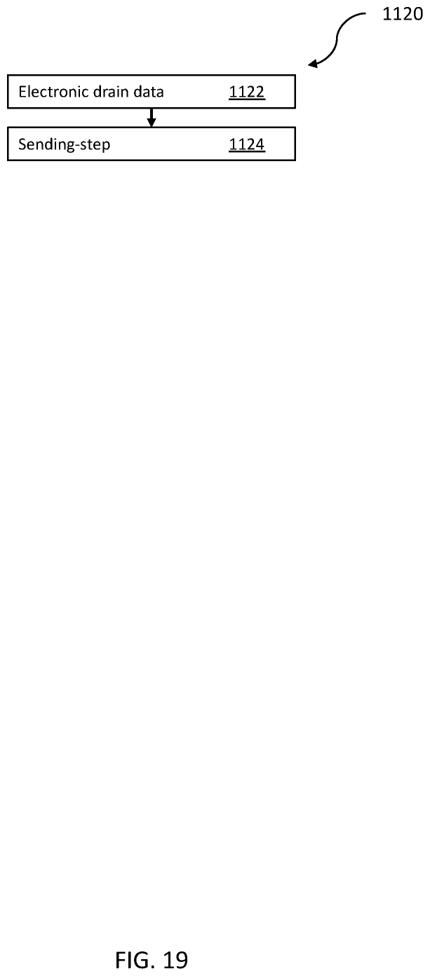

In some aspects, and with reference to FIG. 18, the present disclosure comprises a method 1100 for an automated body fluid drain control system 102 to accept an electronic drain order 1080 from a third-party system 1082, the method comprising the third-party system 1082 having a connection 1102 to the automated body fluid drain control system 102, and wherein the automated body fluid drain control system 102 first, in a receiving-step 1104 an electronic drain order 1080 from the third-party system 1082, whereafter the automated body fluid drain control system 102 validates 1106 that the electronic drain order 1080 came from the third-party system 1082 that was or is validated, the automated body fluid drain control system 102 processes 1108 the electronic drain order 1080, the automated body fluid drain control system 102 thereafter confirms 1110 that the electronic drain order 1080 is within the parameters of the automated body fluid drain control system 102 to perform, thereafter the electronic drain order 1080 configures 1112 the automated body fluid drain control system 102, and the automated body fluid drain control system 102 executes 1114 the electronic drain order 1080. In some aspects, and with reference to FIG. 19, the present disclosure comprises a method 1120 for sending electronic drain data 1122 to a third-party system 1082 from an automated body fluid drain control system 102, the method comprising, with an automated body fluid drain control system 102 configured to monitor physical processes and record actual drain data versus desired drain behaviors, and an interoperable server system 1010 that can accept drain data from an automated body fluid drain control system 102, and a third-party system 1082 that can accept a plurality of electronic drain data 1122, wherein a plurality of electronic drain data 1122 is received by the interoperable server system 1010 from the automated body fluid drain control system 102. In some aspects, the method 1100 may include a sending-step 1124, wherein the automated body fluid drain control system 102 sends a plurality of electronic drain data 1122 to a third-party system 1082.

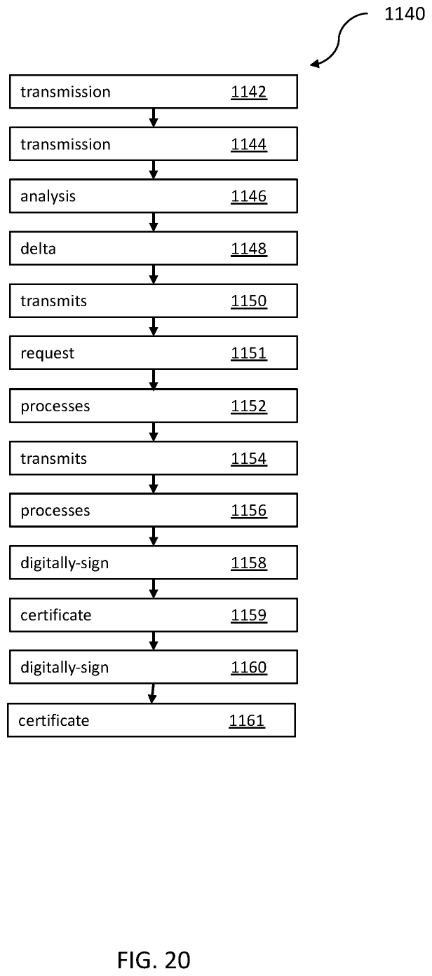

In some aspects, and with reference to FIG. 20, the present disclosure comprises a method 1140 for comparing drain protocol safety libraries that is approved in a drain protocol safety library editor 904 (DPSLE) and the drain protocol safety library 900 (active DPSL) on an automated body fluid drain control system 102, wherein the method 1140 comprises transmission 1142 from the automated body fluid drain control system 102 to the drain protocol safety library editor 904 of a request for the most recent drain protocol safety library 900; transmission 1144 from the drain protocol safety library editor 904 to the automated body fluid drain control system 102 of the most recent known drain protocol safety library 900; analysis 1146 by the automated body fluid drain control system 102 to determine if there is a delta 1148 requiring action by the automated body fluid drain control system 102, and if there is the delta 1148, the automated body fluid drain control system 102 transmits 1150 a request 1151 for the updated drain protocol safety library 900 to the drain protocol safety library editor 904; the drain protocol safety library editor 904 processes 1152 the request 1151 and transmits 1154 the updated drain protocol safety library 900 to the automated body fluid drain control system 102; and the automated body fluid drain control system 102 processes 1156 and stores the updated drain protocol safety library 900. The drain protocol safety library 900 may be stored and transferred in a database file format. The drain protocol safety library 900 may be transferred as a compact, row-oriented delta change and/or as a complete binary encoded file. The drain protocol safety library 900 may be compressed prior to transmission. The automated body fluid drain control system 102 and/or the drain protocol safety library editor 904 may digitally-sign 1158 the request 1151 with a certificate 1159 to ensure the authenticity of the drain protocol safety library 900. The drain protocol safety library editor 904 may digitally-sign 1160 the request 1151 with a certificate 1161 to ensure the authenticity of the drain protocol safety library 900. The automated body fluid drain control system 102 may send the request 1151 on a periodic schedule. The automated body fluid drain control system 102 may send the request 1151 based on a detectable event. In another aspect of the method 1140, the drain protocol safety library editor 904 may request the currently active drain protocol safety library 900 from the automated body fluid drain control system 102.

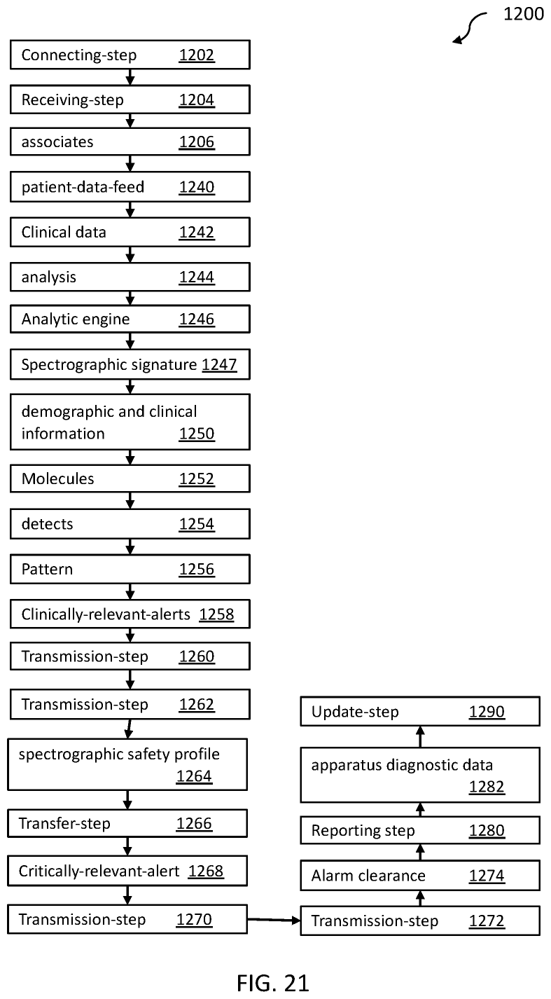

In some aspects, and with reference to FIG. 21, the present disclosure comprises a method 1200 for collecting a plurality of demographic and clinical information 1250 about a patient 800, or about more than one patient, and associating the plurality of demographic and clinical information 1250 with an automated body fluid drain control system 102 in use in conjunction with a third-party system 1082, the method 1200 comprising a connecting-step 1202 of the automated body fluid drain control system 102 to the third-party system 1082; wherein the automated body fluid drain control system 102 receives, in a receiving-step 1204, a patient-data-feed 1240 from the third-party system 1082, wherein the patient-data-feed 1240 comprises a plurality of clinical data 1242; and wherein a user 890 associates 1206 the automated body fluid drain control system 102 to the patient-data-feed 1240 through a process which may be manual. Thereafter, the automated body fluid drain control system 102 provides the plurality of clinical data 1242 back to a data platform 140; and thereafter the data platform 140 stores the demographic and clinical information 1250 associated with the patient 800, and the plurality of clinical data 1242, including any spectral signatures. The plurality of clinical data 1242 may be processed at any later time. The plurality of clinical data 1242 may be stored and transmitted in an encrypted format, and/or the plurality of clinical data 1242 may be compressed using digital compression algorithms. The plurality of clinical data 1242 may be anonymized for storage and/or transmission. The plurality of clinical data 1242 may be transferred to an analytic engine 1246 for processing. The plurality of clinical data 1242 may be analyzed by the analytic engine 1246 in an analysis 1244 may comprise anonymized data elements, of n number of dimensions of data elements, also referred to as the anonymized N-dimensional data elements, from the combination or intersection of the clinical data 1242 and the demographic and clinical information 1250 acquired from the third-party system 1082, and stored in the data platform 140. In some aspects, the clinical data 1242 can be grouped into statistically likely treatment parameters such that any particular instructions for an automated body fluid drain control system 102, including but not limited to configurations, a drain storage protocol 910, or other instructions or programming can be validated to exist or existing within a given standard deviation for the patient population. In some aspects, the clinical data 1242 may be used by the automated body fluid drain control system 102 to detect compounds, medications, molecules, or other spectrographic signatures in CSF fluid, such that the analytic engine 1246 may detect an unknown molecule, medication, compound, or a spectrographic signature 1247, and/or the analysis 1244 may show changes in the spectrographic signature 1247 over time during the course of treatment of a patient 800 in a definable time period, including but not limited to the entire course of treatment. The drainage data together with the patient data feed and the clinical data may be referred to as “the data”. The analysis 1244 detects 1254, in some aspects of the present disclosure, the presence or concentration of any of a set of molecules 1252, including but not limited to oxyhaemoglobin, bilirubin, methaemglobin, glucose, proteins, or medications. When the analysis 1244 detects 1254 the presence or concentration of any of a set of molecules 1252, the detection may be grouped into a normative pattern 1256 for a given population of patients 800, such that the pattern 1256 can be used to differentiate the spectrographic signature 1247 of a patient 800 that is normal over the course of treatment from the spectrographic signature 1247 of a patient 800 that is abnormal over the course of treatment. In some aspects, the treatments can be grouped into outcome groups that provide clinical insight into the most likely outcomes of the current patient 800 treatment, In some aspects, clinically-relevant-alerts 1258 that are detected can be transferred to the data platform 140; such clinically-relevant-alerts 1258 may be detected by the automated body fluid drain control system 102 and/or by other elements of the systems or methods disclosed herein. In some aspects, one or more of the clinically-relevant-alerts 1258 may be transmitted in a transmission-step 1260 to a third-party system 1082 and/or to the automated body fluid drain control system 102 for notification and/or presentation to medical providers or any user 890. In some aspects, the treatment patterns, spectrographic signature 1247, and groupings are transferred to the data platform 140 for real-time analysis, retrospective analysis, or other analysis that may be conducted over any period of time, wherein any such analysis may be of automated body fluid drain control system 102 operations. In some aspects, one or more of the treatment patterns, spectrographic signature 1247, and groupings may be transmitted in a transmission-step 1262 to a third-party system 1082 and/or to the automated body fluid drain control system 102 for notification and/or presentation to medical providers or any user 890. In some aspects, one or more of the treatment patterns, spectrographic signature 1247, and groupings may be transferred to the data platform 140, which stores the one or more of the treatment patterns, spectrographic signature 1247, and groupings into a spectrographic safety profile 1264. The spectrographic safety profile 1264 may be transferred in a transfer-step 1266 to the automated body fluid drain control system 102 for real-time analysis, retrospective analysis, or other analysis that may be conducted over any period of time, wherein any such analysis may be of drainage operations. In some aspects, the spectrographic safety profile 1264 may trigger a critically-relevant alert 1268 which may be transmitted in a transmission-step 1270 to a third-party system 1082 and/or to the automated body fluid drain control system 102 as a notification to a user 890. The automated body fluid drain control system 102 may comprise a user interface 150 that enables configuration of alerts that should be transferred to a user 890 or any medical provider. In some aspects, the user interface 150 enables review of the normative signature-patterns 144 by trained medical providers, and/or by a user 890. The user interface 150 may enable review, including but not limited to real-time review, of alerts that have been detected. In some aspects, a dismissal of an alarm related to a clinically-relevant-alerts 1258 on any device, including but not limited to a third-party system 1082, an analytic engine 1246, a data platform 140, or an automated body fluid drain control system 102 transmits, in a in a transmission-step 1272, a notification of alarm clearance 1274 to all of the other foregoing devices, for common alarm management.

In some aspects, the automated body fluid drain control system 102 can report, in a reporting-step 1280 apparatus diagnostic data 1282 to facilitate maintenance of the automated body fluid drain control system 102. In some aspects, automated body fluid drain control system 102 can be remotely updated, in an update-step 1290, without physical interaction from the user 890.

Certain aspects of the present invention were described above. From the foregoing it will be seen that this invention is one well adapted to attain all the ends and objects set forth above, together with other advantages, which are obvious in and inherent to the inventive apparatus disclosed herein. It will be understood that certain features and sub-combinations are of utility and may be employed without reference to other features and sub-combinations. It is expressly noted that the present invention is not limited to those aspects described above, but rather the intention is that additions and modifications to what was expressly described herein are also included within the scope of the invention. Moreover, it is to be understood that the features of the various aspects described herein are not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

1. An automated body fluid drain control system, the system comprising:
a first controllable flow means having a variant number of states including open to drain, partially open to drain, closed to drain, open to sample, partially open to sample and closed to sample such that it is possible for multiple states to be active concurrently;
a measuring device that r ion to s an amount of body fluid being drained; and
a second controllable flow means having an open and closed state.

2. The system of claim 1, wherein the system further comprises a collection chamber of variable size.

3. The system of claim 2, wherein the system further comprises a vent that connects the collection chamber to open air; and the system further comprises a filter.

4. The system of claim 1 wherein the system calculates a volumetric fluid flow on a periodic basis and adjusts the first controllable flow means to reduce or increase the volumetric fluid flow to fit uniformly within a calculated drainage volume desired for a time period.

5. The system of claim 1 wherein the first controllable flow means is connected to a patient for draining body fluids by gravity.

6. The system of claim 1 wherein the second controllable flow means is connected to an output device for purposes of collecting the body fluids.

7. The system of claim 1 wherein the body fluid is cerebrospinal fluid.

8. The system of claim 1 including a monitor system that indicates an alarm when the body fluids cannot or do not generate a volumetric fluid flow to a flow volume that is requested or desired.

9. The system of claim 8, wherein the monitor system indicates an alarm when the system is not functioning.

10. The system of claim 1 wherein a multi-state valve may be manually operated.

11. The system of claim 1 wherein the system further comprises a spectral analysis port.

12. The system of claim 1 wherein the system further comprises a machine-readable identifier.

13. The system of claim 1 wherein the system further comprises a plurality of drain cassettes.

14. The system of claim 13, wherein each of the plurality of drain cassettes is operated with single-handed insertion into the system, and/or single-handed removal from the system.

15. The system of claim 1 wherein the system further comprises a spectral analysis port and a plurality of spectrophotometric sensors and a plurality of light sources, wherein the plurality of spectrophotometric sensors and the plurality of light sources are capable of generating and sensing light with wavelengths of approximately 250 nm-approximately 1900 nm in or from the spectral analysis port.

16. A method of use of the system of claim 1, the method comprising analysis of the actively draining fluid in quantities which may include minute quantities and in very small time increments.

17. The method of claim 16 including, in the method, data processing and storage on the system such that each spectrophotometric signature can be performed, analyzed and stored on the device even when not in contact with a data platform.

18. The method of claim 17 including, in the method, data indexing against cassette identification, drainage session and/or information about a patient.

19. The method of claim 18 including, in the method, an ability to transfer a plurality of spectrophotometric signatures to the data platform when connected.

20. The method of claim 19 including, in the method, signature plurality of normative signature-patterns stored on the system.

21. The method of claim 20 including, in the method, an ability to download normative signature-patterns to be stored on the system.

22. The method of claim 21 including alerting a user when signatures deviate from the normative signature-patterns.

23. The method of claim 22 where an infusion test is monitored and analyzed for change in drainage fluid spectrographic signature to determine an amount of dilution, if any, and when and after what volume of drained cerebrospinal fluid has the cerebrospinal fluid returned to a pre-infusion level.
